(19) United States
(12) Patent Application Publication
(10) Pub. No.: US 2023/0256039 A1
(43) Pub. Date: Aug. 17, 2023

(54) COMPOSITIONS AND METHODS TO INCREASE PRODUCTION OF HYDROXYTYROSOL

(71) Applicant: Church & Dwight Co., Inc., Princeton, NJ (US)

(72) Inventors: Noah Paul Zimmerman, Chapel Hill, NC (US); Amy Wescott, Rubicon, WI (US)

(73) Assignee: Church & Dwight Co., Inc., Princeton, NJ (US)

(21) Appl. No.: 18/165,127

(22) Filed: Feb. 6, 2023

Related U.S. Application Data
(60) Provisional application No. 63/307,436, filed on Feb. 7, 2022.

(51) Int. Cl. A61K 35/747; A61K 31/7048

(52) U.S. Cl. CPC A61K 35/747; A61K 31/7048

(57) ABSTRACT

Compositions and methods for converting oleuropein to hydroxytyrosol using Lactobacillus pentosus strain OL79 or active variants thereof are provided. The composition can comprise oleuropein or oleuropein-containing substrate such as olives or an extract thereof. Methods for increasing an amount of hydroxytyrosol and providing antioxidant activity, anti-inflammatory activity, anti-platelet aggregation function, cardiovascular protective effects, and/or therapeutic effects on diseases or conditions associated with oxidative stress, inflammation, or platelet aggregation are also provided.

### RELATED APPLICATIONS

This application claims the benefit of priority to U.S. Provisional Application No. 63/307,436, filed on Feb. 7, 2022, the content of which is incorporated herein by reference in its entirety.

### FIELD OF THE INVENTION

The invention relates to microbial compositions for increasing the production of hydroxytyrosol from oleuropein.

### BACKGROUND OF THE INVENTION

The health benefits of a diet rich in plants, fruits and vegetables are due not only to the vitamins, minerals and fiber found in the plants but also to compounds such as polyphenols. Polyphenols such as oleuropein, found in olives and olive oil, are known for providing health benefits. However, multiple studies demonstrate that many of these polyphenol compounds are poorly absorbed into the body. The majority of benefits may come from the smaller, more well absorbed bacterial metabolites, such as hydroxytyrosol rather than from the parent compound.

Smaller polyphenols, such as hydroxytyrosol can exert health benefits through antioxidant and anti-inflammatory activity, anti-platelet aggregation, and vasodilatory effects. For example, hydroxytyrosol has been shown to have beneficial effects on cardiovascular health, which is partially due to the ability to reduce reactive oxygen species (ROS). Hydroxytyrosol has been shown to increase nuclear protein levels of fork-head transcription factor 3a (FOXO3a), which directly increases the expression of anti-oxidant enzymes and can contribute to a reduction in ROS. In addition, hydroxytyrosol has been shown to inhibit cyclooxygenase-2 (COX-2) and thromboxanes and to inhibit platelet aggregation, the first step in atherosclerosis.

The majority of hydroxytyrosol in olives comes from the break-down (e.g., hydrolysis) of oleuropein during ripening of the plant. While hydroxytyrosol can be found in olive oil, the amount depends greatly on the quality of the oil. Accordingly, compositions and methods capable of metabolizing oleuropein to hydroxytyrosol could offer important health and commercial advantages.

### BRIEF SUMMARY OF THE INVENTION

Compositions and methods for converting oleuropein to hydroxytyrosol using Lactobacillus pentosus strain OL79 or active variants thereof are provided. Hydroxytyrosol is a more absorbable metabolite of oleuropein. Once absorbed into the plasma, hydroxytyrosol exerts various health (e.g., cardiovascular health) promoting effects including antioxidant activity, anti-inflammatory activity, anti-platelet aggregation function, and vasodilatory effects. Methods for increasing hydroxytyrosol amount or providing the above health promoting effects are also provided. The composition can comprise oleuropein or oleuropein-containing substrate such as olives or an extract thereof.

In one aspect, provided herein is a bacterial strain composition comprising a bacterial cell of Lactobacillus pentosus strain OL79 or an active variant thereof, wherein said Lactobacillus pentosus strain OL79 cell is present at about 10⁶ CFU/gram to about 10¹⁰ CFU/gram or at about 10⁶ CFU/ml to about 10¹⁰ CFU/ml. In some embodiments, the bacterial strain composition further comprises oleuropein. In some embodiments, the bacterial strain composition comprises at least one plant, plant part, or extract thereof comprising said oleuropein. In some embodiments, said at least one plant, plant part, or extract thereof comprises an olive plant, olive plant part, or extract thereof. In some embodiments, an effective amount of said bacterial strain composition increases conversion of oleuropein to hydroxytyrosol relative to the absence of said effective amount of said bacterial strain composition.

In one aspect, the present disclosure provides a bacterial strain composition comprising oleuropein and a bacterial strain, said bacterial strain comprising a bacterial cell of Lactobacillus pentosus strain OL79 or an active variant thereof. In some embodiments, the bacterial strain composition further comprises at least one plant, plant part, or extract thereof comprising said oleuropein. In some embodiments, said at least one plant, plant part, or extract thereof comprises olive plant, olive plant part, or extract thereof. In some embodiments, an effective amount of said bacterial strain increases conversion of oleuropein to hydroxytyrosol relative to the absence of said effective amount of said bacterial strain.

In further embodiments of any of the preceding aspects and embodiments, bacterial strain composition comprises a capsule, gel, paste, cream, ointment, tablet, powder, or liquid.

In one aspect, the present disclosure provides a pharmaceutical composition comprising a bacterial strain comprising a bacterial cell of Lactobacillus pentosus strain OL79 or an active variant thereof, and a pharmaceutically acceptable carrier that is not naturally-occurring with said bacterial cell. In some embodiments, the pharmaceutical composition further comprises oleuropein. In some embodiments, the pharmaceutical composition comprises at least one plant, plant part, or extract thereof comprising said oleuropein. In some embodiments, said at least one plant, plant part, or extract thereof comprises olive plant, olive plant part, or extract thereof. In some embodiments, said pharmaceutical composition comprises a capsule, gel, paste, cream, ointment, tablet, powder, or liquid.

In one aspect, the present disclosure provides a method of increasing hydroxytyrosol production, said method comprising contacting oleuropein with an effective amount of a bacterial strain composition comprising a bacterial cell of Lactobacillus pentosus strain OL79 or an active variant thereof. In some embodiments, said method comprises contacting at least one plant, plant part, or extract thereof comprising said oleuropein with said effective amount of said bacterial strain. In some embodiments, said at least one plant, plant part, or extract thereof comprises olive plant, olive plant part, or extract thereof.

In one aspect, the present disclosure provides a method of increasing an amount of hydroxytyrosol in a subject, said method comprising administering to a subject an effective amount of a bacterial strain composition comprising a bacterial cell of Lactobacillus pentosus strain OL79 or an active variant thereof. In some embodiments, said subject is administered oleuropein. In some embodiments, said bacterial strain composition further comprises said oleuropein. In some embodiments, said subject is administered at least one plant, plant part, or extract thereof comprising said oleuropein. In some embodiments, said at least one plant, plant part, or extract thereof comprises olive plant, olive plant part, or extract thereof. In some embodiments, said bacterial composition is administered orally or dermally.

In further embodiments of the any of the methods of the present disclosure, said effective amount of said bacterial strain composition comprises about 10⁶ CFU/gram to about 10¹⁰ CFU/gram or about 10⁶ CFU/ml to about 10¹⁰ CFU/ml of said Lactobacillus pentosus strain OL79 or the active variant thereof. In some embodiments, said bacterial strain composition comprises a capsule, gel, paste, cream, ointment, tablet, powder, or liquid.

In one aspect, the present disclosure provides a method for reducing or preventing oxidative stress and/or inflammation in a subject, said method comprising administering to said subject an effective amount of a pharmaceutical composition comprising a bacterial strain comprising a bacterial cell of Lactobacillus pentosus strain OL79 or an active variant thereof, and a pharmaceutically acceptable carrier.

In one aspect, the present disclosure provides a method for inhibiting platelet aggregation in a subject, said method comprising administering to said subject an effective amount of a pharmaceutical composition comprising a bacterial strain comprising a bacterial cell of Lactobacillus pentosus strain OL79 or an active variant thereof, and a pharmaceutically acceptable carrier.

In one aspect, the present disclosure provides a method for treating a disease or a condition associated with oxidative stress, inflammation, and/or platelet aggregation in a subject, said method comprising administering to said subject an effective amount of a pharmaceutical composition comprising a bacterial strain comprising a bacterial cell of Lactobacillus pentosus strain OL79 or an active variant thereof, and a pharmaceutically acceptable carrier. In some embodiments, the disease or the condition is a cardiovascular disease, a metabolic disease, an inflammatory disease, a neurodegenerative disease, or a cancer.

In some embodiments of the methods of the preceding aspects and embodiments, said subject is administered oleuropein. In some embodiments, said pharmaceutical composition further comprises said oleuropein. In some embodiments, said subject is administered at least one plant, plant part, or extract thereof comprising said oleuropein. In some embodiments, said at least one plant, plant part, or extract thereof comprises olive plant, olive plant part, or extract thereof.

In some embodiments the methods of the preceding aspects and embodiments, an amount of hydroxytyrosol is increased in the subject relative to the absence of said effective amount of said pharmaceutical composition. In some embodiments, said pharmaceutical composition is administered orally or dermally. In some embodiments, said effective amount of said pharmaceutical composition comprises about 10⁶ CFU/gram to about 10¹⁰ CFU/gram or about 10⁶ CFU/ml to about 10¹⁰ CFU/ml of said Lactobacillus pentosus strain OL79 or the active variant thereof. In some embodiments, said pharmaceutical composition comprises a capsule, gel, paste, cream, ointment, tablet, powder, or liquid.

In one aspect, the present disclosure provides a method of reducing oxidative stress, reducing inflammation, and/or reducing platelet aggregation, said method comprising contacting a cell with an effective amount of a bacterial strain composition comprising oleuropein and a bacterial cell of Lactobacillus pentosus strain OL79 or an active variant thereof. In some embodiments, said cell comprises a vascular cell, a cardiac cell, a blood cell, an intestinal cell, a liver cell, a pancreatic cell, a kidney cell, a lung cell, an adipose cell, a neural cell, or a skin cell. In some embodiments, the cell is contacted with an increased amount of hydroxytyrosol relative to the absence of said effective amount of said bacterial strain composition.

### BRIEF DESCRIPTION OF THE DRAWINGS

FIG. 1 shows progressive Mauve alignment of the genome of Lactobacillus pentosus strain OL79 and related strains.

FIG. 2 provides a high performance liquid chromatography (HPLC) chromatograph of an oleuropein conversion reaction. The black line represents the chromatograph of oleuropein (retention time at about 7.3 minutes) and its metabolites in minimal media after an 18 hour anaerobic incubation with Lactobacillus pentosus strain OL79. The peak at about 1.0 minute is consistent with the retention time of hydroxytyrosol.

### DETAILED DESCRIPTION

The present disclosure now will be described more fully hereinafter. The disclosure may be embodied in many different forms and should not be construed as limited to the embodiments set forth herein; rather, these embodiments are provided so that this disclosure will satisfy applicable legal requirements. Like numbers refer to like elements throughout. As used in this specification and the claims, the singular forms “a,” “an,” and “the” include plural references unless the context clearly dictates otherwise.

### I. Overview

Provided herein are compositions and methods comprising a strain of Lactobacillus pentosus OL79 or an active variant thereof, which is capable of converting oleuropein to hydroxytyrosol.

Polyphenols such as oleuropein, naturally found in plants, plant parts, and extracts thereof e.g., olive plants, olive leaves, olive fruits, and olive oil provide health benefits. However, many of these polyphenol compounds are poorly absorbed into the body (Marín et al. 2015 BioMed Res. Int. 2015; 1-18). The majority of benefits may come from the smaller, more well absorbed metabolites (e.g., bacterial metabolites) or fragments, such as hydroxytyrosol, rather than from the parent compound (Selma et al. 2009 J. Agric. Food Chem. 57; 6485-6501). As used herein, “oleuropein” refers to a polyphenol comprising the following structure (Structure I):

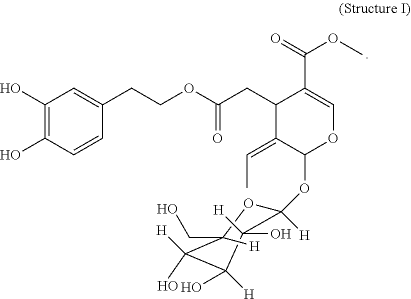 [SMILES: C/C=C1\C(OC2OC(CO)C(O)C(O)C2O)OC=C(C(=O)OC)C1CC(=O)OCCc1ccc(O)c(O)c1]

Oleuropein can be broken down (e.g., hydrolyzed) into hydroxytyrosol and eloneic acid. As used herein, “hydroxytyrosol” refers to an amphipathic phenol with a phenyl-ethyl-alcohol structure (Structure II):

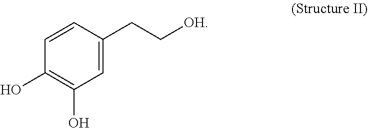 [SMILES: OCCc1ccc(O)c(O)c1]

Oleuropein and hydroxytyrosol can be naturally found in a plant, a plant part, or an extract thereof, such as a soluble fraction of olive oil.

Smaller polyphenols, such as hydroxytyrosol can exert significant benefits to health through antioxidant activity, anti-inflammatory activity, anti-platelet aggregation, and vasodilatory effects (Tejada et al. 2017 Current Drug Targets 18:13). Hydroxytyrosol has been shown to have an ability to reduce reactive oxygen species (ROS). The antioxidant effect of hydroxytyrosol can be exerted via its capacity of scavenging oxidant chemical species, as well as on the ability to stimulate the synthesis and activity of antioxidant enzymes, e.g., superoxide dismutase (SOD), catalase (CAT), endothelial nitric oxide synthase (eNOS), glutathione peroxidase (GPx), and glutathione reductase (GR) (D'Angelo et al. 2020 Cells 9:1932). Hydroxytyrosol can increase nuclear protein levels of fork-head transcription factor 3a (FOXO3a), which directly increases the expression of anti-oxidant enzymes and can contribute to a reduction in ROS (Vilaplana-Perez et al. 2014 Frontiers in Nutrition 18:1-11). Hydroxytyrosol has also been shown to inhibit platelet aggregation in part by inhibiting cyclooxygenase-2 (COX-2) and thromboxanes (e.g., thromboxane A2, thromboxane B2). Platelet aggregation is the first step in atherosclerosis (Bulotta et al 2014 J. Translat. Med. 12:219). Hydroxytyrosol has also been shown to prevent or reduce inflammation in part by reducing expression or function of cyclooxygenase-2, thromboxane A2, or thromboxane B2, vascular adhesion molecule-1 (VCAM-1), or other pro-inflammatory molecules (e.g., inducible nitric oxide synthase, prostaglandin E2, prostaglandin E2 synthase, IL-1alpha, IL-1b, IL-6, IL-12, TNF-alpha, CXCL10/IP-10, CCL2/MPC-1, MIP-1beta, matrix metaloproteinase-9) (Bulotta et al 2014 J. Translat. Med. 12; 219). Having multiple health promoting effects including antioxidant function, anti-inflammatory function, and anti-platelet aggregation function, hydroxytyrosol has been reported to have cardiovascular protective effects, anti-metabolic disease effects, neuroprotective effects, would healing effects, anti-microbial effects, and anti-tumor effects (Vilaplana-Pérez et al. 2014 Frontiers Nutrition 1:18; 1-11).

The majority of hydroxytyrosol in olives comes from the hydrolysis of the parent compound, oleuropein, during ripening of the plant (Robles-Almazan et al. 2018 Food Research Int. 105:654-667). While hydroxytyrosol can be found in olive oil, the amount depends greatly on the quality of the oil (Vilaplana-Perez et al. 2014 Frontiers in Nutrition 18:1-11).

The identification of a bacterial strain that is safe for ingestion and is capable of surviving in the gastrointestinal tract to produce hydroxytyrosol in the intestine by biotransforming oleuropein allows for the compositions and methods of increasing an amount of hydroxytyrosol in vivo, ex vivo, or in vitro. Oleuropein can be ingested through a diet consisting of olive plant, olive plant part, or an extract thereof, e.g., olive oil. The bacterial strain composition of the present disclosure can also be ingested e.g., as a probiotic composition. This approach allows for the capture of hydroxytyrosol from ingested food (e.g., olives, olive oil). The hydroxytyrosol can then be absorbed into the body immediately after production.

The bacterial strains described herein (and in some embodiments oleuropein or a plant, plant part or extract thereof comprising oleuropein, alone or in combination with the bacterial strains) can be administered orally as a dietary supplement in a powder, a capsule, a gel, a paste, or a tablet and in combinations with food items or alone, dermally in a cream, a gel, a paste, an ointment, a powder, a solution, a suspension, or an emulsion, in oils, as a suppository, in lubricants, sachets, topically along with oleuropein in a matrix, or via other administration routes.

In some embodiments, compositions and methods further comprise the provision of oleuropein that can be acted upon by the presently disclosed bacterial strains. In some of these embodiments, the oleuropein is a naturally-occurring oleuropein, for example, within an olive plant.

Though not wishing to be bound by any particular theory, it is believed that polyphenols and simple phenols (e.g., hydroxytyrosol) may provide protection against oxidative and inflammatory stress, such as disturbances of systems that protect cells against oxidative damage, heat shock, and disturbances caused by protein misfolding.

The presently disclosed bacterial strains can be used as probiotics. The term “probiotics” has been defined by the Food and Agriculture Organization of the United Nations (FAO) and World Health Organization (WHO) as live microorganisms which when administered in adequate amounts confer a health benefit on the host. Probiotics include beneficial bacteria that when consumed or otherwise administered to the gastrointestinal tract improve the health of the subject. In some embodiments, the beneficial bacteria colonize the gut, allowing for a more persistent beneficial effect.

### II. Bacterial Strains

Bacterial strains are provided which can be used to convert oleuropein to hydroxytyrosol. Such bacterial strains include Lactobacillus pentosus strain OL79 or an active variant thereof. Cell populations comprising Lactobacillus pentosus strain OL79 or an active variant thereof are provided, as well as any preparation thereof. Thus, bacterial strains and/or compositions provided herein comprise as an active ingredient a cell population of Lactobacillus pentosus strain OL79 or an active variant thereof.

In addition to the ability to convert oleuropein to hydroxytyrosol, the bacterial strains disclosed herein may encode secondary metabolite biosynthetic genes that offer heath promoting benefits. For example, Lactobacillus pentosus strain OL79 or an active variant thereof may encode a type III polyketide synthase, a pediocin, and bovicin-like peptide. A type III polyketide synthase can produce an antibiotic (e.g., tetracycline, macroride), an anti-cancer drug (e.g., epothilone B), an immunosuppressant (e.g., sirolimus), or an anticholesterol drug (e.g., statin). A pediocin can be used as an antimicrobial. A bovicin-like peptide can provide a lantibiotic function.

The term “isolated” encompasses a bacterium, spore, or other entity or substance, that has been (1) separated from at least some of the components with which it was associated when initially produced (whether in nature or in an experimental setting), and/or (2) produced, prepared, purified, and/or manufactured by the hand of man. Isolated bacteria may be separated from at least about 10%, about 20%, about 30%, about 40%, about 50%, about 60%, about 70%, about 80%, about 90%, or more of the other components with which they were initially associated.

As used herein, a substance is “pure” if it is substantially free of other components. The terms “purify,” “purifying” and “purified” refer to a bacterium, spore, or other material that has been separated from at least some of the components with which it was associated either when initially produced or generated (e.g., whether in nature or in an experimental setting), or during any time after its initial production. A bacterium or spore or a bacterial population or a spore population may be considered purified if it is isolated at or after production, such as from a material or environment containing the bacterium or bacterial population or spore, and a purified bacterium or bacterial population or spore may contain other materials up to about 10%, about 20%, about 30%, about 40%, about 50%, about 60%, about 70%, about 80%, about 90%, or above about 90% and still be considered purified. In some embodiments, purified bacteria or spores and bacterial populations or spore populations are more than about 80%, about 85%, about 90%, about 91%, about 92%, about 93%, about 94%, about 95%, about 96%, about 97%, about 98%, about 99%, or more than about 99% pure. In specific embodiments, a culture of bacteria contains no other bacterial species in quantities to be detected by normal bacteriological techniques.

By “population” is intended a group or collection that comprises two or more (i.e., 10, 100, 1,000, 10,000, 1×10⁶, 1×10⁷, or 1×10⁸ or greater), for example, of a given bacterial strain. Various compositions are provided herein that comprise a population of at least one bacterial strain or a mixed population of individuals from more than one bacterial strain. In specific embodiments, the population of Lactobacillus pentosus strain OL79, an active variant thereof, or combination thereof comprises a concentration of at least about 10⁴ CFU/ml to about 10¹² CFU/ml, about 10⁵ CFU/ml to about 10¹² CFU/ml, about 10⁶ CFU/ml to about 10¹² CFU/ml, about 10⁷ CFU/ml to about 10¹² CFU/ml, about 10⁸ CFU/ml to about 10¹² CFU/ml, about 10⁹ CFU/ml to about 10¹² CFU/ml, about 10¹⁰ CFU/ml to about 10¹² CFU/ml, about 10¹¹ CFU/ml to about 10¹² CFU/ml, about 10⁵ CFU/ml to about 10¹¹ CFU/ml, about 10⁵ CFU/ml to about 10¹¹ CFU/ml, about 10⁶ CFU/ml to about 10¹¹ CFU/ml, about 10⁷ CFU/ml to about 10¹¹ CFU/ml, about 10⁸ CFU/ml to about 10¹¹ CFU/ml, about 10⁹ CFU/ml to about 10¹¹ CFU/ml, about 10¹⁰ CFU/ml to about 10¹¹ CFU/ml, about 10⁵ CFU/ml to about 10¹⁰ CFU/ml, about 10⁶ CFU/ml to about 10¹⁰ CFU/ml, about 10⁷ CFU/ml to about 10¹⁰ CFU/ml, about 10⁸ CFU/ml to about 10¹⁰ CFU/ml, or about 10⁹ CFU/ml to about 10¹⁰ CFU/ml. In other embodiments, the concentration of the bacterial strain provided herein, an active variant thereof, or combination thereof comprises or consists of at least about 10⁴ CFU/ml, at least about 10⁵ CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, or at least about 10¹² CFU/ml. In specific embodiments, the population of Lactobacillus pentosus strain OL79, an active variant thereof, or combination thereof comprises a concentration of at least about 10⁴ CFU/g to about 10¹² CFU/g, about 10⁵ CFU/g to about 10¹² CFU/g, about 10⁶ CFU/g to about 10¹² CFU/g, about 10⁷ CFU/g to about 10¹² CFU/g, about 10⁸ CFU/g to about 10¹² CFU/g, about 10⁹ CFU/g to about 10¹² CFU/g, about 10¹⁰ CFU/g to about 10¹² CFU/g, about 10¹¹ CFU/g to about 10¹² CFU/g, about 10⁵ CFU/g to about 10¹¹ CFU/g, about 10⁵ CFU/g to about 10¹¹ CFU/g, about 10⁶ CFU/g to about 10¹¹ CFU/g, about 10⁷ CFU/g to about 10¹¹ CFU/g, about 10⁸ CFU/g to about 10¹¹ CFU/g, about 10⁹ CFU/g to about 10¹¹ CFU/g, about 10¹⁰ CFU/g to about 10¹¹ CFU/g, about 10⁵ CFU/g to about 10¹⁰ CFU/g, about 10⁶ CFU/g to about 10¹⁰ CFU/g, about 10⁷ CFU/g to about 10¹⁰ CFU/g, about 10⁸ CFU/g to about 10¹⁰ CFU/g, or about 10⁹ CFU/g to about 10¹⁰ CFU/g. In other embodiments, the concentration of the bacterial strain provided herein, active variant thereof, or combination thereof comprises or consists of at least about 10⁴ CFU/g, at least about 10⁵ CFU/g, at least about 10⁶ CFU/g, at least about 10⁷ CFU/g, at least about 10⁸ CFU/g, at least about 10⁹ CFU/g, at least about 10¹⁰ CFU/g, at least about 10¹¹ CFU/g, or at least about 10¹² CFU/g.

### A. Active Variants of a Bacterial Strain

Further provided are active variants of Lactobacillus pentosus strain OL79 that retain the ability to convert (e.g., hydrolyze) oleuropein to hydroxytyrosol. An active variant includes a strain having all of the identifying characteristics of the recited strain. A “strain of the invention” includes active variants thereof.

By “modified bacterial strain” is intended a population wherein the strain has been modified (by selection and/or transformation) to have one or more additional traits of interest. Modified bacterial strains can be made through genetic engineering techniques and such engineered or recombinant bacterial strains grown to produce a modified population of bacterial strains. A recombinant bacterial strain can be produced by introducing polynucleotides into the bacterial host cell by transformation or by otherwise altering the native bacterial chromosome sequence, including but not limited to, gene editing approaches. Methods for transforming microorganisms are known and available in the art. See, generally, Hanahan, D. (1983) Studies on transformation of Escherichia coli with plasmids J. Mol. Biol. 166, 557-77; Seidman, C. E. (1994) In: Current Protocols in Molecular Biology, Ausubel, F. M. et al. eds., John Wiley and Sons, NY; Choi et al. (2006) J. Microbiol. Methods 64:391-397; Wang et al. 2010. J. Chem. Technol. Biotechnol. 85:775-778. Transformation may occur by natural uptake of naked DNA by competent cells from their environment in the laboratory. Alternatively, cells can be made competent by exposure to divalent cations under cold conditions, by electroporation, by exposure to polyethylene glycol, by treatment with fibrous nanoparticles, or other methods well known in the art.

Active variants of the various bacteria provided herein can be identified by employing, for example, methods that determine the sequence identity relatedness between the 16S ribosomal RNA, methods to identify groups of derived and functionally identical or nearly identical strains include Multi-locus sequence typing (MLST), concatenated shared genes trees, Whole Genome Alignment (WGA), Average Nucleotide Identity, and MinHash (Mash) distance metric.

In one aspect, the active variants of the bacterial strain(s) disclosed herein include strains that are closely related to the bacterial strain(s) disclosed herein by employing the Bishop MLST method of organism classification as defined in Bishop et al. (2009) BMC Biology 7(1)1741-7007-7-3. Thus, in specific embodiments, an active variant of Lactobacillus pentosus strain OL79 includes a bacterial strain that falls within at least an 80%, 85%, 86%, 87%, 88%, 89%, 90%, 91%, 92%, 93%. 94%, 95%, 96%, 97%, 98%, 98.5%, 98.8%, 99%, 99.1%, 99.2%, 99.3%, 99.4%, 99.5%, 99.6%, 99.7%, 99.8%, or 99.9% sequence cut off employing the Bishop method of organism classification as set forth in Bishop et al. (2009) BMC Biology 7(1)1741-7007-7-3, which is herein incorporated by reference in its entirety. Active variants of the bacteria identified by such methods will retain the ability to convert oleuropein to hydroxytyrosol.

In some embodiments, the active variant of the bacterial strain(s) disclosed herein include strains that are closely related to the disclosed strain(s) disclosed herein on the basis of the Average Nucleotide Identity (ANI) method of organism classification. ANI (see, for example, Konstantinidis, K. T., et al., (2005) PNAS USA 102(7):2567-72; and Richter, M., et al., (2009) PNAS 106(45):19126-31) and variants (see, for example, Varghese, N.J., et al., Nucleic Acids Research (Jul. 6, 2015): gkv657) are based on summarizing the average nucleotides shared between the genomes of strains that align in WGAs. Thus, in specific embodiments, an active variant of bacterial strain Lactobacillus pentosus OL79 disclosed herein includes a bacterial strain that falls within at least a 90%, 95%, 96%, 97%, 97.5%, 98%, 98.5%, 98.8%, 99%, 99.5%, 99.8%, or 99.9% sequence cut off employing the ANI method of organism classification as set forth in Konstantinidis, K. T., et al., (2005) PNAS USA 102(7):2567-72, which is herein incorporated by reference in its entirety. Active variants of the bacteria identified by such methods will retain the ability to convert oleuropein to hydroxytyrosol.

In particular embodiments, the active variants of the isolated bacterial strain(s) disclosed herein include strain(s) that are closely related to the bacterial strain(s) disclosed herein on the basis of 16S rDNA sequence identity. See Stackebrandt E, et al., “Report of the ad hoc committee for the re-evaluation of the species definition in bacteriology,” Int J Syst Evol Microbiol. 52(3):1043-7 (2002) regarding use of 16S rDNA sequence identity for determining relatedness in bacteria. In an embodiment, the active variant of Lactobacillus pentosus strain OL79 is at least 95%, 96%, 97%, 97.5%, 98%, 98.5%, 98.8%, 99%, 99.5%, 99.8%, or 99.9% identical to Lactobacillus pentosus strain OL79 on the basis of 16S rDNA sequence identity. Active variants of the bacteria identified by such methods will retain the ability to convert oleuropein to hydroxytyrosol.

The MinHash (Mash) distance metric is a comparison method that defines thresholds for hierarchical classification of microorganisms at high resolution and requires few parameters and steps (Ondov et al. (2016) Genome Biology 17:132). The Mash distance estimates the mutation rate between two sequences directly from their MinHash sketches (Ondov et al. (2016) Genome Biology 17:132). Mash distance strongly corresponds to Average Nucleotide Identity method (ANI) for hierarchical classification (See, Konstantinidis, K. T. et al. (2005) PNAS USA 102(7):2567-72, herein incorporated by reference in its entirety). That is, an ANI of 97% is approximately equal to a Mash distance of 0.03, such that values put forth as useful classification thresholds in the ANI literature can be directly applied with the Mash distance.

Active variants of the bacterial strain(s) disclosed herein include strains that are closely related to the bacterial strain(s) disclosed herein on the basis of the Minhash (Mash) distance between complete genome DNA sequences. Thus, in specific embodiments, an active variant of Lactobacillus pentosus strain OL79 includes bacterial strains having a genome within a Mash distance of less than about 0.015 to the disclosed strains. In other embodiments, an active variant of Lactobacillus pentosus strain OL79 includes a distance metric of less than about 0.001, 0.0025, 0.005, 0.010, 0.015, 0.020, 0.025, or 0.030. A genome, as it relates to the Mash distance includes both bacterial chromosomal DNA and bacterial plasmid DNA. In other embodiments, the active variant of a bacterial strain has a genome that is above a Mash distance threshold to the disclosed strains that is greater than dissimilarity caused by technical variance. In further instances, the active variant of a bacterial strain has a genome that is above a Mash distance threshold to the disclosed strains that is greater than dissimilarity caused by technical variance and has a Mash distance of less than about 0.015. In other instances, the active variant of a bacterial strain has a genome that is above a Mash distance threshold to the disclosed strains that is greater than dissimilarity caused by technical variance and has a Mash distance of less than about 0.001, 0.0025, 0.005, 0.010, 0.015, 0.020, 0.025, or 0.030.

As used herein, “above technical variation” means above the Mash distance between two strains caused by errors in the genome assemblies provided the genomes being compared were each DNA sequenced with at least 20× coverage with the Illumina HiSeq 2500 DNA sequencing technology and the genomes are at least 99% complete with evidence for contamination of less than 2%. While 20× coverage is an art recognized term, for clarity, an example of 20× coverage is as follows: for a genome size of 5 megabases (MB), 100 MB of DNA sequencing from the given genome is required to have 20× sequencing coverage on average at each position along the genome. There are many suitable collections of marker genes to use for genome completeness calculations including the sets found in Campbell et al. (2013) PNAS USA 110(14):5540-45, Dupont et al. (2012) ISMEJ 6:1625-1628, and the CheckM framework (Parks et al. (2015) Genome Research 25:1043-1055); each of these references is herein incorporated in their entirety. Contamination is defined as the percentage of typically single copy marker genes that are found in multiple copies in the given genome sequence (e.g. Parks et al. (2015) Genome Research 25:1043-1055); each of these references is herein incorporated in their entirety. Completeness and contamination are calculated using the same collection of marker genes. Unless otherwise stated, the set of collection markers employed in the completeness and contamination assay is those set forth in Campbell et al. (2013) PNAS USA 110(14):5540-45, herein incorporated by reference.

Exemplary steps to obtain a distance estimate between the genomes in question are as follows: (1) Genomes of sufficient quality for comparison must be produced. A genome of sufficient quality is defined as a genome assembly created with enough DNA sequence to amount to at least 20× genome coverage using Illumina HiSeq 2500 technology. The genome must be at least 99% complete with contamination of less than 2% to be compared to the claimed microbe's genome. (2) Genomes are to be compared using the Minhash workflow as demonstrated in Ondov et al. (2016) Genome Biology 17:132, herein incorporated by reference in its entirety. Unless otherwise stated, parameters employed are as follows: “sketch” size of 1000, and “k-mer length” of 21. (3) Confirm that the Mash distance between the two genomes is less than 0.001, 0.0025, 0.005, 0.010, 0.015, 0.020, 0.025, or 0.030. Using the parameters and methods stated above, a Mash distance of 0.015 between two genomes means the expected mutation rate is 0.015 mutations per homologous position. Active variants of the bacteria identified by such methods will retain the ability to convert oleuropein to hydroxytyrosol.

### B. Methods of Cultivating Bacterial Strains

Populations or cultures of Lactobacillus pentosus strain OL79 or an active variant thereof can be produced by cultivation of the bacterial strain. Cultivation can be started by scaling-up a seed culture. This involves repeatedly and aseptically transferring the culture to a larger and larger volume to serve as the inoculum for the fermentation, which can be carried out in large stainless steel fermenters in medium containing proteins, carbohydrates, and minerals necessary for optimal growth of the strain. Non-limiting exemplary medium for the Lactobacillus strains is de Man Rogosa Sharpe (MRS) (de Man et al. 1960 J. Appl. Bacteriol. 23:130; herein incorporated by reference in its entirety). After the bacterial inoculum is added to the fermentation vessel, the temperature and agitation are controlled to allow maximum growth. Once the culture reaches a maximum population density, the culture is harvested by separating the cells from the fermentation medium. This separation is commonly performed by centrifugation.

The concentration of the bacterial culture can be measured from any sample of fermentation broth or bacterial strain composition. A colony forming unit (CFU) is the viable cell count of a sample resulting from standard microbiological plating methods. The term is derived from the fact that a single cell when plated on appropriate medium will grow and become a viable colony in the agar medium. Since multiple cells may give rise to one visible colony, the term colony forming unit can be a more useful unit measurement than cell number.

The various compositions and formulations disclosed herein can comprise an amount of Lactobacillus pentosus strain OL79 or an active variant thereof, i.e., cells of Lactobacillus pentosus strain OL79 or an active variant thereof. Such an amount can comprise a concentration of Lactobacillus pentosus strain OL79 or an active variant thereof of at least about 10⁴ CFU/g to about 10¹² CFU/g, about 10⁵ CFU/g to about 10¹² CFU/g, about 10⁶ CFU/g to about 10¹² CFU/g, about 10⁷ CFU/g to about 10¹² CFU/g, about 10⁸ CFU/g to about 10¹² CFU/g, about 10⁹ CFU/g to about 10¹² CFU/g, about 10¹⁰ CFU/g to about 10¹² CFU/g, about 10¹¹ CFU/g to about 10¹² CFU/g, about 10⁵ CFU/g to about 10¹¹ CFU/g, about 10⁵ CFU/g to about 10¹¹ CFU/g, about 10⁶ CFU/g to about 10¹¹ CFU/g, about 10⁷ CFU/g to about 10¹¹ CFU/g, about 10⁸ CFU/g to about 10¹¹ CFU/g, about 10⁹ CFU/g to about 10¹¹ CFU/g, about 10¹⁰ CFU/g to about 10¹¹ CFU/g, about 10⁵ CFU/g to about 10¹⁰ CFU/g, about 10⁶ CFU/g to about 10¹⁰ CFU/g, about 10⁷ CFU/g to about 10¹⁰ CFU/g, about 10⁸ CFU/g to about 10¹⁰ CFU/g, or about 10⁹ CFU/g to about 10¹⁰ CFU/g. In other embodiments, the concentration of Lactobacillus pentosus strain OL79 or active variant thereof comprises or consists of at least about 10⁴ CFU/g, at least about 10⁵ CFU/g, at least about 10⁶ CFU/g, at least about 10⁷ CFU/g, at least about 10⁸ CFU/g, at least about 10⁹ CFU/g, at least about 10¹⁰ CFU/g, at least about 10¹¹ CFU/g, or at least about 10¹² CFU/g. Another such an amount can comprise a concentration of Lactobacillus pentosus strain OL79 or an active variant thereof of at least about 10⁴ CFU/ml to about 10¹² CFU/ml, about 10⁵ CFU/ml to about 10¹² CFU/ml, about 10⁶ CFU/ml to about 10¹² CFU/ml, about 10⁷ CFU/ml to about 10¹² CFU/ml, about 10⁸ CFU/ml to about 10¹² CFU/ml, about 10⁹ CFU/ml to about 10¹² CFU/ml, about 10¹⁰ CFU/ml to about 10¹² CFU/ml, about 10¹¹ CFU/ml to about 10¹² CFU/ml, about 10⁵ CFU/ml to about 10¹¹ CFU/ml, about 10⁵ CFU/ml to about 10¹¹ CFU/ml, about 10⁶ CFU/ml to about 10¹¹ CFU/ml, about 10⁷ CFU/ml to about 10¹¹ CFU/ml, about 10⁸ CFU/ml to about 10¹¹ CFU/ml, about 10⁹ CFU/ml to about 10¹¹ CFU/ml, about 10¹⁰ CFU/ml to about 10¹¹ CFU/ml, about 10⁵ CFU/ml to about 10¹⁰ CFU/ml, about 10⁶ CFU/ml to about 10¹⁰ CFU/ml, about 10⁷ CFU/ml to about 10¹⁰ CFU/ml, about 10⁸ CFU/ml to about 10¹⁰ CFU/ml, or about 10⁹ CFU/ml to about 10¹⁰ CFU/ml. In other embodiments, the concentration of Lactobacillus pentosus strain OL79 or an active variant thereof comprises or consists of at least about 10⁴ CFU/ml, at least about 10⁵ CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, or at least about 10¹² CFU/ml.

### III. Compositions Comprising a Bacterial Strain

The bacterial strains provided herein (i.e., cells of Lactobacillus pentosus strain OL79 or an active variant thereof) can be formulated as a paste (e.g., cell paste), a powder, a capsule, a tablet, a granule, a cell pellet, dust, a slurry, aqueous or oil based liquid products, gel, and the like.

Common bacterial strain compositions, such as probiotic preparations, are liquid solutions and concentrates or lyophilized powders for resuspension, which can be enclosed in a capsule, vial, or pouch. Such formulations will comprise the bacterial strains provided herein or an active variant thereof, in addition to carriers and other agents. As used herein, the term “carrier” refers to an inert compound that is compatible with any other ingredients in the formulation and is not deleterious to the active compound (i.e., bacterial strains) or a subject that the formulation is administered thereto. Suitable carriers can be added to improve recovery, efficacy, or physical properties and/or to aid in packaging and administration. Such carriers may be added individually or in combination. Non-limiting examples of carriers include proteins, carbohydrates, fats, enzymes, vitamins, immune modulators, oligosaccharides, milk replacers, minerals, amino acids, coccidiostats, acid-based products, medicines (such as antibiotics), other probiotics, and/or prebiotics. Common carriers include cellulose, sugar, glucose, lactose, whey powder, or rice hulls. In specific embodiments, the carrier does not naturally occur with the bacterial strain provided herein.

The carrier(s) may comprise about 30% weight per weight, weight per volume, or volume per volume, of the final composition. In some embodiments, the carrier(s) may comprise about 40%, about 50%, about 60%, about 70%, about 80%, about 90%, about 91%, about 92%, about 93%, about 94%, about 95%, about 96%, about 97%, about 98%, about 98.5%, about 99.0%, about 99.5%, or about 99.9% weight per weight, weight per volume, or volume per volume of the final composition.

In some embodiments, the bacterial strain composition comprises a pharmaceutical composition wherein the bacterial strains provided herein are formulated as a pharmaceutical composition along with a pharmaceutically acceptable carrier. Such pharmaceutically acceptable carriers are known in the art and include an inert vehicle, adjuvants, preservatives etc., which are well known. In some embodiments, the pharmaceutically acceptable carrier comprises one that is not naturally-occurring (i.e., not found in nature). In particular embodiments, the pharmaceutically acceptable carrier is a naturally-occurring carrier that is not found with a bacterial strain of the invention in the native environment of the bacterial strain (i.e., a pharmaceutically acceptable carrier that is not naturally-occurring with a bacterial cell of a bacterial strain of the invention). Such pharmaceutical compositions can be prepared in accordance with known techniques. See, e.g., Remington, The Science and Practice of Pharmacy (21st ed. 2005).

In some embodiments, feed and/or food compositions can be prepared by combining a formulated bacterial strain of the invention with typical animal feed and/or food or drink ingredients. A formulated bacterial strain of the invention can be used for the preparation of animal feed or food products or beverages, and/or may be added to drinking and/or rearing water. In other embodiments, the compositions of the present invention are feed, food and/or drink additives that are added to a subject's feed, food, drinking water or beverage prior to ingestion.

As used herein, “animal feed” includes any animal feed blend known in the art, including rapeseed meal, cottonseed meal, soybean meal, cornmeal, barley, wheat, silage, and haylage.

The bacterial strains provided herein can be formulated as a food composition such as a dietary supplement, a functional food, a medical food or a nutritional product as long as the required effect is achieved, i.e. conversion of oleuropein to hydroxytyrosol. Said food compositions may be chosen from the group consisting of beverages, yogurts, juices, ice creams, breads, biscuits, crackers, cereals, health bars, spreads, and nutritional products. The food composition may further comprise a carrier material, wherein said carrier material is chosen from the group consisting of lactic acid fermented foods, fermented dairy products, resistant starch, dietary fibers, carbohydrates, proteins and glycosylated proteins.

In some embodiments, the bacterial strain composition disclosed herein is formulated as a liquid formulation or a solid formulation. When the bacterial strain composition (e.g., pharmaceutical composition) is a solid formulation, it may be formulated as a tablet, a sucking tablet, a chewing tablet, a chewing gum, a capsule, a sachet, a powder, a granule, a coated particle, a coated tablet, an enterocoated tablet, an enterocoated capsule, a melting strip, or a film. When the bacterial strain composition (e.g., pharmaceutical composition) is a liquid formulation, it may be formulated as an oral solution, a suspension, an emulsion or syrup. The composition may further comprise a carrier material independently selected from, but not limited to, the group consisting of vegetables, lactic acid fermented foods, fermented dairy products, resistant starch, dietary fibers, carbohydrates, proteins, and glycosylated proteins. In specific embodiments, the bacterial strain composition is formulated into a formulation that does not naturally occur and that improves a property of the bacterial strain composition. For example, the bacterial strain composition can be formulated to improve stability, improve activity, improve storage time, improve the range of acceptable storage temperatures or conditions, improve formulation or processing efficiency, or improve bacterial health.

Additional beneficial microbes may be combined with a bacterial strain of the invention into a formulated product. Alternatively, additional formulated probiotics may be combined or mixed with a formulated bacterial strain of the invention into a feed or food composition, into drinking water, or into a pharmaceutical composition. Alternatively, the additional probiotic may be administered at a different time. These additional beneficial microbes may be selected from species of Saccharomyces, species of Bacillus such as Bacillus subtilis, Bacillus licheniformis, Bacillus lentus, Bacillus pumilus, Bacillus laterosporus, Bacillus coagulans, Bacillus alevi, Bacillus cereus, Bacillus clausii, Bacillus coagulans, Bacillus inaquosorum, Bacillus mojavensis, Bacillus velezensis, Bacillus vallismortis, Bacillus amyloliquefaciens, Bacillus atropheus, Bacillus altitudinis, Bacillus safensis, Bacillus alcalophilus, Bacillus badius, or Bacillus thurigiensis; from species of Enterococcus such as Enterococcus faecium; from species of Clostridium such as Clostridium butyricum; from species of Lactococcus such as Lactococcus lactis or Lactoccus cremoris; from species of Bifidobacterium such as Bifidobacterium adolescentis, Bifidobacterium animalis, Bifidobacterium bifidum, Bifidobacterium infantis, Bifidobacterium longum, Bifidobacterium pseudolongum, or Bifidobacterium thermophilum; from species of Lactobacillus such as Lactobacillus alactosus, Lactobacillus alimentarius, Lactobacillus amylovorans, Lactobacillus amylophilus, Lactobacillus amylovorans, Lactobacillus acidophilus, Lactobacillus agilis, Lactobacillus animalis, Lactobacillus batatas, Lactobacillus bavaricus, Lactobacillus bifermentans, Lactobacillus bidifus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus curvatus, Lactobacillus coprohilus, Lactobacillus delbrueckii, Lactobacillus fermentum, Lactobacillus gasseri, Lactobacillus jugurti, Lactobacillus kefir, Lactobacillus lactis, Lactobacillus leichmannii, Lactobacillus mali, Lactobacillus malefermentans, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus tolerans, Lactobacillus torquens, Lactobacillus ruminis, Lactobacillus sake, Lactobacillus saliverius, Lactobacillus sharpeae, Lactobacillus sobrius, Lactobacillus trichodes, Lactobacillus vaccinostercus, Lactobacillus viridescens, Lactobacillus vitulinus, Lactobacillus xylosus, Lactobacillus yamanashiensis, or Lactobacillus zeae; from species of Megasphaera such as Megasphaera elsdenil; from species of Prevotella such as Prevotella bryantii; from species of Pediococcus such as Pediococcus acidilactici, or Pediococcus pentosaceus; from species of Streptococcus such as Streptococcus cremoris, Streptococcus discetylactis, Streptococcus faecium, Streptococcus lactis, Streptococcus thermophilus, or Streptococcus intermedius; or from species of Propionibacterium such as Propionibacterium freudenreichii, Propionibacterium acidipropionici, Propionibacterium jensenii, Propionibacterium thoenii, Propionibacterium australiense, or Propionibacterium avidum, and/or a combination thereof.

Compositions of the invention may also include prebiotics, which may be combined or mixed with a formulated bacterial strain of the invention into a feed or food composition, into drinking water, or into a pharmaceutical composition. Prebiotics are food ingredients that are not readily digestible by enzymes endogenous to the gut (such as those expressed by the animal or those expressed by the resident gut microbiome) and that selectively stimulate the growth and activity of selected groups of intestinal microorganisms that confer beneficial effects upon their host. Typically, it is beneficial microorganism populations that benefit from the presence of prebiotic compounds. Prebiotics can consist of oligosaccharides and other small molecules that serve as metabolic substrates for growth of beneficial microbes. Common prebiotics include galacto-oligosaccharides, fructo-oligosaccharides, inulin, isomalto-oligosaccharies, gentio-oligosaccharides, lactilol, lactosucrose, lactulose, xylosucrose, glycosyl sucrose, pyrodextrins, soybean oligosaccharides, guar gum, locust bean gum, arabinan, galactan, pectins, and pectic polysaccharides. While many diverse microbes inhabit the intestinal tract of a host organism, prebiotic compounds are only utilized by the beneficial microbes and lead to a selective enhancement of the beneficial microbe population. A formulation that includes both prebiotics and probiotics may be known as a “synbiotic”.

In certain embodiments, the bacterial strain composition comprises oleuropein. In some embodiments, the bacterial strain composition comprises oleuropein at an amount of about 1 μg to about 1 g, including but not limited to about 1 μg to about 2 μg, about 300 μg to about 100 mg, about 350 μg to about 30 mg, about 1 μg, about 2 μg, about 5 μg, about 10 μg, about 20 μg, about μg, about 50 μg, about 100 μg, about 200 μg, about 300 μg, about 400 μg, about 500 μg, about 600 μg, about 700 μg, about 800 μg, about 900 μg, about 1 mg, about 2 mg, about 5 mg, about 10 mg, about 20 mg, about 25 mg, about 50 mg, about 100 mg, about 200 mg, about 300 mg, about 400 mg, about 500 mg, about 600 mg, about 700 mg, about 800 mg, about 900 mg, and about 1 g. In certain embodiments, the bacterial strain composition comprises a plant, plant part or extract thereof that comprises the oleuropein. Oleuropein may be synthetically produced or derived from a natural source. Oleuropein is found in olive plants. Oleuropein is found in abundance in the leaves, fruits (peel, pulp, and seed), and oil produced therefrom of olive plants. An “olive plant” as used herein refers to a plant in the Oleaceae family (i.e., olive family) including but not limited to Olea europaea, other species of the Olea genus, and other genera in the Oleaceae family. Plants comprising oleuropein that can be used for the compositions and methods of the present disclosure include, but are not limited to, Olea europaea (olive), Olea ambrensis, Olea borneensis, Olea brachiata, Olea capensis, Olea capitellata Ridl, Olea caudatilimba, Olea chimanimani, Olea cordatula, Olea dioica, Olea exasperata, Olea gagnepainii, Olea gamblei, Olea hainanensis, Olea javanica, Olea lancea, Olea laxiflora, Olea moluccensis, Olea neriifolia, Olea palawanensis, Olea paniculata, Olea parvilimba, Olea polygama, Olea puberula, Olea rosea, Olea rubrovenia, Olea salicifolia, Olea schliebenii, Olea tetragonoclada, Olea tsoongii, Olea welwitschii, Olea wightiana, Olea woodiana, Olea yuennanensis. Fraxinus excelsior, Fraxinus angustifolia, Fraxinus chinensis, Fraxinus mandshurica var japonica, Syringa josikaea, Syringa vulgaris, Phillyrea latifolia, Ligustrum ovalifolium, Ligustrum vulgare, Jasminum polyanthum, and Osmanthus asiaticus.

In some embodiments, the bacterial strain composition comprises an olive plant, plant part, or extract thereof. Plant sources suitable for use in the methods and compositions disclosed herein may be any part of a plant, including, but not limited to cells, seeds, sprouts, leaves, stalks, roots, flowers, and other plant structures.

Oleuropein can also be obtained from extracts of any of the above-mentioned species of olive plants or plant parts. As used herein, an “extract” of a plant or plant part refers to concentrated preparations comprising a desired active compound derived from the plant or plant part using any method known in the art, including but not limited to solvent extraction, distillation method, pressing, and sublimation (see, e.g., Zhang et al. (2018) Chin Med. 13:20). According to the presently disclosed methods and compositions, the extract (e.g., olive oil) of a plant or plant part can comprise oleuropein, which can be converted to hydroxytyrosol by the composition comprising Lactobacillus pentosus strain OL79 or an active variant thereof described herein.

Oleuropein can be converted to hydroxytyrosol chemically and/or enzymatically in the presence of a bacterial strain composition disclosed herein. Bacterial strain compositions of the present disclosure can comprise an enzymatic function that converts (e.g., hydrolyzes) oleuropein to hydroxytyrosol, e.g., β-glucosidase, hemicellulase, tannase, neutral protease, cellulase, glucoamylase, papain, alkaline protease, amylase, or β-glucanase function. Bacterial strain compositions of the invention can also comprise an enzyme potentiator (i.e., cofactor). Enzyme potentiators may be used to enhance the activity of the oleuropein hydrolyzing enzyme. In some embodiments, the enzyme potentiator comprises ascorbic acid, also known as ascorbate or vitamin C, which can serve as a base catalyst in oleuropein hydrolysis. In some embodiments, an enzyme potentiator such as ascorbic acid can facilitate the conversion reaction to produce hydroxytyrosol to achieve peak absorption in the location needed. The enzyme potentiator may be obtained from a natural source, or it may be produced synthetically.

In some embodiments, the bacterial strain composition comprises a concentration of the bacterial strain (e.g., Lactobacillus pentosus strain OL79 or an active variant thereof) of at least about 10⁴ CFU/g to about 10¹² CFU/g, about 10⁵ CFU/g to about 10¹² CFU/g, about 10⁶ CFU/g to about 10¹² CFU/g, about 10⁷ CFU/g to about 10¹² CFU/g, about 10⁸ CFU/g to about 10¹² CFU/g, about 10⁹ CFU/g to about 10¹² CFU/g, about 10¹⁰ CFU/g to about 10¹² CFU/g, about 10¹¹ CFU/g to about 10¹² CFU/g, about 10⁵ CFU/g to about 10¹¹ CFU/g, about 10⁵ CFU/g to about 10¹¹ CFU/g, about 10⁶ CFU/g to about 10¹¹ CFU/g, about 10⁷ CFU/g to about 10¹¹ CFU/g, about 10⁸ CFU/g to about 10¹¹ CFU/g, about 10⁹ CFU/g to about 10¹¹ CFU/g, about 10¹⁰ CFU/g to about 10¹¹ CFU/g, about 10⁵ CFU/g to about 10¹⁰ CFU/g, about 10⁶ CFU/g to about 10¹⁰ CFU/g, about 10⁷ CFU/g to about 10¹⁰ CFU/g, about 10⁸ CFU/g to about 10¹⁰ CFU/g, or about 10⁹ CFU/g to about 10¹⁰ CFU/g. In other embodiments, the concentration of the bacterial strain provided herein or active variant thereof comprises or consists of at least about 10⁴ CFU/g, at least about 10⁵ CFU/g, at least about 10⁶ CFU/g, at least about 10⁷ CFU/g, at least about 10⁸ CFU/g, at least about 10⁹ CFU/g, at least about 10¹⁰ CFU/g, at least about 10¹¹ CFU/g, or at least about 10¹² CFU/g.

In liquid compositions and formulations, the amount of the bacterial strain or an active variant thereof disclosed herein can comprise a concentration of at least about 10⁴ CFU/ml to about 10¹² CFU/ml, about 10⁵ CFU/ml to about 10¹² CFU/ml, about 10⁶ CFU/ml to about 10¹² CFU/ml, about 10⁷ CFU/ml to about 10¹² CFU/ml, about 10⁸ CFU/ml to about 10¹² CFU/ml, about 10⁹ CFU/ml to about 10¹² CFU/ml, about 10¹⁰ CFU/ml to about 10¹² CFU/ml, about 10¹¹ CFU/ml to about 10¹² CFU/ml, about 10⁵ CFU/ml to about 10¹¹ CFU/ml, about 10⁵ CFU/ml to about 10¹¹ CFU/ml, about 10⁶ CFU/ml to about 10¹¹ CFU/ml, about 10⁷ CFU/ml to about 10¹¹ CFU/ml, about 10⁸ CFU/ml to about 10¹¹ CFU/ml, about 10⁹ CFU/ml to about 10¹¹ CFU/ml, about 10¹⁰ CFU/ml to about 10¹¹ CFU/ml, about 10⁵ CFU/ml to about 10¹⁰ CFU/ml, about 10⁶ CFU/ml to about 10¹⁰ CFU/ml, about 10⁷ CFU/ml to about 10¹⁰ CFU/ml, about 10⁸ CFU/ml to about 10¹⁰ CFU/ml, or about 10⁹ CFU/ml to about 10¹⁰ CFU/ml. In other embodiments, the concentration of the bacterial strain provided herein or active variant thereof comprises or consists of at least about 10⁴ CFU/ml, at least about 10⁵ CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, or at least about 10¹² CFU/ml.

### IV. Methods Comprising a Bacterial Strain

Methods are provided herein for increasing the production of hydroxytyrosol from oleuropein with an effective amount of a bacterial strain composition comprising a bacterial strain of the invention. The methods comprise contacting oleuropein with an effective amount of a bacterial strain composition comprising a bacterial strain of the invention. These methods can be performed in vitro, in vivo, or ex vivo. When performed in vivo, the method comprises administering to a subject an effective amount of a bacterial strain composition of the invention comprising the bacterial strain an active variant thereof.

In specific embodiments, a bacterial strain composition is combined with oleuropein to increase the production of hydroxytyrosol when compared to an appropriate control (e.g., a sample prior to the addition of a bacterial strain composition). As used herein an “effective amount” refers to a quantity or concentration of a bacterial strain composition comprising a bacterial strain of the invention that increases the production of hydroxytyrosol from oleuropein when compared to an appropriate control (e.g., a sample prior to the addition of a bacterial strain composition). In some embodiments, an effective amount of the bacterial strain composition can increase the expression of genes regulated by the fork-head transcription factor 3a (FOXO3a), increase the expression or function of anti-oxidant enzymes [e.g., superoxide dismutase (SOD), catalase (CAT), endothelial nitric oxide synthase (eNOS), glutathione peroxidase (GPx), glutathione reductase (GR)], reduce expression or function of cyclooxygenase-2, thromboxane A2, or thromboxane B2, vascular adhesion molecule-1 (VCAM-1), or other pro-inflammatory molecules (e.g., inducible nitric oxide synthase, prostaglandin E2, prostaglandin E2 synthase, IL-la, IL-1b, IL-6, IL-12, TNF-α, CXCL10/IP-10, CCL2/MPC-1, MIP-1b, matrix metaloproteinase-9) in a cell or a subject, when compared to an appropriate control.

An effective amount of the bacterial strain composition can reduce oxidative stress, reduce production or amount of the reactive oxygen species (ROS) levels or reactive nitrogen species (RNS) levels, prevent or reduce inflammation, produce vasodilation, reduce or inhibit platelet aggregation, or promote would healing in a subject when compared to an appropriate control.

An effective amount of the bacterial strain composition can treat or prevent various diseases and conditions that involve inflammatory process and/or oxidative stress, including inflammatory disorders (e.g., inflammatory bowel disease, Crohn's disease, ulcerative colitis, rheumatoid arthritis, systemic lupus erythematosus, multiple sclerosis, atopic dermatitis), cardiovascular diseases (e.g., atherosclerosis, coronary artery disease, peripheral vascular disease, stroke), metabolic disorders (e.g., diabetes mellitus, glucose intolerance, dyslipidemia), neurodegenerative diseases (e.g., Alzheimer's disease, Parkinson's disease), brain or spinal cord injury, and cancer (e.g., breast cancer, colon cancer, bladder cancer, brain cancer, leukemia, prostate cancer, renal cancer, thyroid cancer).

An effective amount of the bacterial strain composition (e.g., pharmaceutical composition) is determined based on the intended goal. The term “unit dose” refers to a physically discrete unit suitable for use in a subject or agricultural animal, each unit containing a predetermined quantity of the bacterial strain composition calculated to produce the desired response in association with its administration, i.e., the appropriate route and treatment regimen. The quantity to be administered, both according to number of treatments and unit dose, depends on the subject to be treated, the state of the subject, the environmental conditions of the subject, and the result desired. Precise amounts of the bacterial strain composition also depend on the judgment of the practitioner and can be unique to each individual.

In specific embodiments, the effective amount of a bacterial strain, or active variant thereof, disclosed herein is at least about 10⁴ CFU to about 10¹² CFU, about 10⁵ CFU to about 10¹² CFU, about 10⁶ CFU to about 10¹² CFU, about 10⁷ CFU to about 10¹² CFU, about 10⁸ CFU to about 10¹² CFU, about 10⁹ CFU to about 10¹² CFU, about 10¹⁰ CFU to about 10¹² CFU, about 10¹¹ CFU to about 10¹² CFU, about 10⁵ CFU to about 10¹¹ CFU, about 10⁵ CFU to about 10¹¹ CFU, about 10⁶ CFU to about 10¹¹ CFU, about 10⁷ CFU to about 10¹¹ CFU, about 10⁸ CFU to about 10¹¹ CFU, about 10⁹ CFU to about 10¹¹ CFU, about 10¹⁰ CFU to about 10¹¹ CFU, about 10⁵ CFU to about 10¹⁰ CFU, about 10⁶ CFU to about 10¹⁰ CFU, about 10⁷ CFU to about 10¹⁰ CFU, about 10⁸ CFU to about 10¹⁰ CFU, or about 10⁹ CFU to about 10¹⁰ CFU. In other embodiments, the effective amount is at least about 10⁴ CFU, at least about 10⁵ CFU, at least about 10⁶ CFU, at least about 10⁷ CFU, at least about 10⁸ CFU, at least about 10⁹ CFU, at least about 10¹⁰ CFU, at least about 10¹¹ CFU, or at least about 10¹² CFU.

In specific embodiments, the effective amount of a bacterial strain, or active variant thereof, disclosed herein is at least about 10⁴ CFU/gram to about 10¹² CFU/gram, about 10⁵ CFU/gram to about 10¹² CFU/gram, about 10⁶ CFU/gram to about 10¹² CFU/gram, about 10⁷ CFU/gram to about 10¹² CFU/gram, about 10⁸ CFU/gram to about 10¹² CFU/gram, about 10⁹ CFU/gram to about 10¹² CFU/gram, about 10¹⁰ CFU/gram to about 10¹² CFU/gram, about 10¹¹ CFU/gram to about 10¹² CFU/gram, about 10⁵ CFU/gram to about 10¹¹ CFU/gram, about 10⁵ CFU/gram to about 10¹¹ CFU/gram, about 10⁶ CFU/gram to about 10¹¹ CFU/gram, about 10⁷ CFU/gram to about 10¹¹ CFU/gram, about 10⁸ CFU/gram to about 10¹¹ CFU/gram, about 10⁹ CFU/gram to about 10¹¹ CFU/gram, about 10¹⁰ CFU/gram to about 10¹¹ CFU/gram, about 10⁵ CFU/gram to about 10¹⁰ CFU/gram, about 10⁶ CFU/gram to about 10¹⁰ CFU/gram, about 10⁷ CFU/gram to about 10¹⁰ CFU/gram, about 10⁸ CFU/gram to about 10¹⁰ CFU/gram, about 10⁹ CFU/gram to about 10¹⁰ CFU/gram, at least about 10⁴ CFU/gram, at least about 10⁵ CFU/gram, at least about 10⁶ CFU/gram, at least about 10⁷ CFU/gram, at least about 10⁸ CFU/gram, at least about 10⁹ CFU/gram, at least about 10¹⁰ CFU/gram, at least about 10¹¹ CFU/gram, or at least about 10¹² CFU/gram.

In specific embodiments, the effective amount of a bacterial strain, or active variant thereof, disclosed herein is at least about 10⁴ CFU/ml to about 10¹² CFU/ml, about 10⁵ CFU/ml to about 10¹² CFU/ml, about 10⁶ CFU/ml to about 10¹² CFU/ml, about 10⁷ CFU/ml to about 10¹² CFU/ml, about 10⁸ CFU/ml to about 10¹² CFU/ml, about 10⁹ CFU/ml to about 10¹² CFU/ml, about 10¹⁰ CFU/ml to about 10¹² CFU/ml, about 10¹¹ CFU/ml to about 10¹² CFU/ml, about 10⁵ CFU/ml to about 10¹¹ CFU/ml, about 10⁵ CFU/ml to about 10¹¹ CFU/ml, about 10⁶ CFU/ml to about 10¹¹ CFU/ml, about 10⁷ CFU/ml to about 10¹¹ CFU/ml, about 10⁸ CFU/ml to about 10¹¹ CFU/ml, about 10⁹ CFU/ml to about 10¹¹ CFU/ml, about 10¹⁰ CFU/ml to about 10¹¹ CFU/ml, about 10⁵ CFU/ml to about 10¹⁰ CFU/ml, about 10⁶ CFU/ml to about 10¹⁰ CFU/ml, about 10⁷ CFU/ml to about 10¹⁰ CFU/ml, about 10⁸ CFU/ml to about 10¹⁰ CFU/ml, about 10⁹ CFU/ml to about 10¹⁰ CFU/ml, at least about 10⁴ CFU/ml, at least about 10⁵ CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, or at least about 10¹² CFU/ml.

In specific embodiments, the effective amount of the bacterial strain composition comprises the bacterial cell of Lactobacillus pentosus strain OL79 or an active variant thereof at a concentration that does not occur in nature (e.g., at a higher concentration than that found in nature). Such non-naturally occurring effective amount (e.g., at a non-naturally occurring concentration) of the bacterial strain can confer the composition the ability to increase conversion of oleuropein to hydroxytyrosol, to reduce oxidative stress, inflammation, and/or platelet aggregation, and/or to treat in a subject cardiovascular disease, a metabolic disease, an inflammatory disease, a neurodegenerative disease, or a cancer, relative to the absence of the non-naturally occurring effective amount (e.g., at a non-naturally occurring concentration) of the bacterial strain composition.

In certain embodiments, a bacterial strain or an active variant thereof disclosed herein is administered to a subject that is also administered (simultaneously or sequentially) an additional beneficial microbe. Additional beneficial microbes, such as those described elsewhere herein, may be combined with a bacterial strain of the invention into a formulated product or the beneficial microbes may be administered separately from (before, during, or after) a bacterial strain of the invention.

In some embodiments, a bacterial strain or an active variant thereof disclosed herein is administered to a subject that is also administered (simultaneously or sequentially) a prebiotic. Prebiotics, such as those described elsewhere herein, may be combined with a bacterial strain of the invention into a formulated product or the prebiotic(s) may be administered separately from (before, during, or after) a bacterial strain of the invention.

In particular embodiments, a bacterial strain or an active variant thereof disclosed herein is administered to a subject that is also administered (simultaneously or sequentially) oleuropein or a plant, plant part or extract thereof comprising oleuropein. The plant, plant part or extract thereof comprising oleuropein can be olive plants, olive plant parts, or extract thereof. Oleuropein, plants, plant parts, or extracts thereof, such as those described elsewhere herein, may be combined with a bacterial strain of the invention into a formulated product or the oleuropein, plant, plant part, or extract thereof, may be administered separately from (before, during, or after) a bacterial strain of the invention. In some of these embodiments, oleuropein is administered in an amount ranging about 1 μg to about 1 g, including but not limited to about 1 μg to about 2 μg, about 300 μg to about 100 mg, about 350 μg to about 30 mg, about 1 μg, about 2 μg, about 5 μg, about 10 μg, about μg, about 25 μg, about 50 μg, about 100 μg, about 200 μg, about 300 μg, about 400 μg, about 500 μg, about 600 μg, about 700 μg, about 800 μg, about 900 μg, about 1 mg, about 2 mg, about 5 mg, about 10 mg, about 20 mg, about 25 mg, about 50 mg, about 100 mg, about 200 mg, about 300 mg, about 400 mg, about 500 mg, about 600 mg, about 700 mg, about 800 mg, about 900 mg, and about 1 g.

In those embodiments wherein the beneficial microbe, prebiotic, synbiotic, oleuropein, plant, plant part, or extract thereof is administered separately from a bacterial strain of the invention (e.g., Lactobacillus pentosus strain OL79 and an active variant thereof), the bacterial strain composition may be administered before, during, or after the beneficial microbe, probiotic, prebiotic, synbiotic, oleuropein, plant, plant part, or extract thereof. The bacterial strain of the invention and the additional component (e.g., beneficial microbe, probiotic, prebiotic, synbiotic, oleuropein, plant, plant part, or extract thereof) can be administered to a subject within minutes (e.g., 1, 2, 5, 10, 15, 30, 45 minutes), hours (e.g., 1, 2, 3, 4, 5, 6, 7, 8, 9, 10, 12, 15, 20 hours), or days (e.g., 1, 2, 3, 4, 5, 6, 7 days) of each other.

As used herein, the bacterial strain composition can be used to increase the production of hydroxytyrosol from oleuropein in any subject when compared to an appropriate control (e.g., a sample from the subject prior to administration of a bacterial strain composition). By “subject” is intended animals. In specific embodiments, subjects are mammals, e.g., primates or humans. In other embodiments, subjects include domestic animals, such as a feline or canine, or agricultural animals, such as a ruminant, horse, swine, poultry, or sheep.

As used herein, an “increase in” or “increasing” hydroxytyrosol production comprises any statistically significant increase in the level of hydroxytyrosol when compared to an appropriate control (e.g., a corresponding sample not contacted with a bacterial strain composition). Such increases can include, for example, at least a 5%, 10%, 20%, 30%, 40%, 50%, 60%, 70%, 80%, 90%, 100%, 150%, 200% or greater increase in the hydroxytyrosol level when compared to an appropriate control. Such increases can also include, for example, at least about a 3%-15%, 10%-25%, 20% to 35%, 30% to 45%, 40%-55%, 50%-65%, 60%-75%, 70%-85%, 80%-95%, 90%-105%, 100%-115%, 105%-120%, 115%-130%, 125%-150%, 140%-160%, 155%-500% or greater increase in the hydroxytyrosol level when compared to an appropriate control. Hydroxytyrosol levels can be measured in a bacterial composition. In some embodiments, the level of hydroxytyrosol can be measured in a sample taken from a subject, or measured directly in the subject. In specific embodiments, the level of hydroxytyrosol is measured in a vascular sample, an intestinal sample, a colon sample, a blood sample (e.g., plasma, serum, whole blood, fraction thereof), a urine sample, other body fluid samples, or a fecal sample taken from a subject.

In specific embodiments, an increase in hydroxytyrosol production is measured in reference to the level of hydroxytyrosol in a proper control sample. As used herein, a proper control includes but is not limited to, the level of hydroxytyrosol in the absence of a bacterial strain of the invention, the level of hydroxytyrosol in a corresponding sample that does not comprise a bacterial strain of the invention, the level of hydroxytyrosol from a subject that was not administered a bacterial strain composition comprising a bacterial strain of the invention, the level of hydroxytyrosol in a sample from the subject prior to administration of a bacterial strain composition comprising a bacterial strain of the invention, or the level of hydroxytyrosol in a standardized sample from a subject that was not administered a bacterial strain composition comprising a bacterial strain of the invention. One of skill in the art would be able to identify proper controls in order to measure an increase in the level of hydroxytyrosol. In specific embodiments, an increase in the level of hydroxytyrosol in the presence of oleuropein can be used to measure the conversion of oleuropein to hydroxytyrosol.

Thus, the presently disclosed methods comprise increasing hydroxytyrosol production by contacting oleuropein with an effective amount of a bacterial strain composition comprising a bacterial strain of the invention when compared to an appropriate control, which can be the level of hydroxytyrosol in the absence of an effective amount of a bacterial strain composition, the level of hydroxytyrosol in a corresponding sample that does not comprise a bacterial strain of the invention, the level of hydroxytyrosol from a subject that was not administered a bacterial strain composition comprising a bacterial strain of the invention, the level of hydroxytyrosol in a sample from the subject prior to administration of a bacterial strain composition comprising a bacterial strain of the invention, or the level of hydroxytyrosol in a standardized sample from a subject that was not administered a bacterial strain composition comprising a bacterial strain of the invention.

Levels of oleuropein, hydroxytyrosol, or any derivative thereof can then be quantified spectroscopically using high performance liquid chromatography (HPLC), or mass spectrometry. Oleuropein, hydroxytyrosol, or any derivative thereof can be positively identified by comparison of the retention time and UV spectrum to authentic chemical standards. See, for example, Ye et al. (2002), which is herein incorporated by reference in its entirety.

The bacterial strain composition can be administered to a subject based on standard techniques known in the art for administration to the particular type of subject and in the environment in which the subject receives the bacterial strain composition. When administered to a human, the bacterial strain composition may be a liquid formulation or a solid formulation. The bacterial strain composition can be administered mucosally via oral administration (e.g., as a dietary supplement, alone or in combination with food items; or as a pharmaceutical composition), nasal administration, or rectal administration, for example; or administered parenterally, including but not limited to subcutaneous administration, cutaneous administration, dermal administration, percutaneous administration, transdermal administration, or any method that allows a bacterial strain of the invention to come into contact with oleuropein. For example, a bacterial strain of the invention can be combined with oleuropein or a plant extract or plant part comprising oleuropein and applied onto the skin in a form of a cream, a gel, a paste, an ointment, a powder, a solution, a suspension, or an emulsion.

In some embodiments of the invention, the method comprises administration of multiple doses of the bacterial composition to a subject. The method may comprise administration of 1, 2, 3, 4, 5, 6, 7, 8, 9, 10, 15, 20, 25, 30, 35, 40, or more effective doses of a bacterial strain composition comprising Lactobacillus pentosus strain OL79 or an active variant thereof as described herein. In some embodiments, doses are administered over the course of 1 day, 2 days, 3 days, 4 days, 5 days, 6 days, 7 days, 10 days, 14 days, 21 days, 30 days, or more than 30 days. The frequency and duration of administration of multiple doses of the compositions is such as to increase the production of hydroxytyrosol when compared to an appropriate control. It will also be appreciated that the effective amount or dosage of a bacterial strain composition may increase or decrease over the course of a particular treatment. Changes in dosage may result and become apparent from the results of diagnostic assays for detecting hydroxytyrosol level known in the art and described herein.

As used herein, “treatment” or “treating” refers to therapeutic (e.g., curing, healing, alleviating, relieving, altering, remedying, ameliorating, improving, or affecting the condition or the symptoms of a subject) and preventative effects. The prophylactic administration (wherein a bacterial strain of the invention is administered in advance of occurrence of diseases, conditions, diagnosis thereof, or symptoms thereof) serves to prevent or attenuate any subsequent symptom. When provided therapeutically, the bacterial strain composition can be provided at, shortly after, or after the diagnosis or an onset of a symptom associated with a disease or a condition. The therapeutic administration of the substance may serve to attenuate any actual symptom, slow or stop progression of the disease or condition, slow or stop recurrence of the disease or condition, or cure the disease or condition.

### A. Methods of Reducing or Preventing Oxidative Stress and Enhancing Antioxidant Activity

Compositions and methods are provided herein for reducing or preventing oxidative stress and providing an antioxidant effect in vitro, ex vivo, or in vivo by contacting a cell with, or administering to a subject, a bacterial strain composition comprising a bacterial strain disclosed herein, and (in some embodiments) oleuropein or a plant, plant part, or extract thereof comprising oleuropein. Any cell may be contacted with a bacterial composition of the invention to reduce or prevent oxidative stress or to enhance antioxidant activity when compared to an appropriate control (e.g., a cell without contact with the bacterial strain composition). In some embodiments, the cell is a vascular cell (e.g., a vascular endothelial cell), a cardiac cell (e.g., a cardiomyocyte), a blood cell (e.g., a platelet, a leukocyte), an intestinal cell (e.g., an intestinal epithelial cell), a liver cell, a pancreatic cell, a kidney cell, a lung cell, an adipose cell, a neural cell, or a skin cell.

In some embodiments, administration of a bacterial strain composition comprising a bacterial strain of the invention can reduce or inhibit oxidative stress or provide an antioxidant effect. In some embodiments, the reduction or prevention of oxidative stress and promotion of antioxidant effect may include or involve decreased amount or production of reactive oxygen species (ROS) or reactive nitrogen species (RNS), such as superoxide anion (O₂⁻), hydrogen peroxide (H₂O₂), hydroxyl radical (OH⁻), and peroxynitrite (ONOO⁻), an increased expression or activity of transcription factors [e.g., fork-head transcription factor 3a (FOXO3a)] that activate antioxidant enzymes, an increased expression or activity of antioxidant enzymes such as superoxide dismutase 1 (SOD1), catalase (CAT), glutathione peroxidase (GPx), glutathione reductase (GR), eNOS, nNOS, and an increased amount or production of reduced glutathione (GSH) and constitutive NO. Hydroxytyrosol provided by the compositions and methods of the present disclosure can exert an antioxidant effect by binding and eradicating ROS and RNS as well as by activating antioxidant enzymes and antioxidant enzyme-activating transcription factors.

As used herein, a reduction of oxidative stress comprises any statistically significant decrease in the level of oxidative stress in vitro, ex vivo, or in vivo when compared to an appropriate control. In some embodiments, contacting a cell with the bacterial strain composition or administering the bacterial strain composition results in a reduction or inhibition of about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or by at least 10%, 15%, 20%, 25%, 30%, 35%, 40%, 45%, 50%, 55%, 60%, 65%, 70%, 75%, 80%, 85%, 90%, 95%, or 100%, in the level of oxidative stress when compared to an appropriate control. Methods to assay for oxidative stress are known and include, for example, assays to measure the levels of DNA/RNA damage, lipid peroxidation, and protein oxidation/nitration. See, for example, Katerji et al. 2019 Oxid Med Cell Longev 1279250.

In some embodiments, contacting a cell with the bacterial strain composition or administering the bacterial strain composition results in a reduction of about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or by at least 10%, 15%, 20%, 25%, 30%, 35%, 40%, 45%, 50%, 55%, 60%, 65%, 70%, 75%, 80%, 85%, 90%, 95%, or 100%, in production or amount, or activity of ROS or RNS. Methods to assay for ROS and RNS are known and include, for example, by using fluorogenic probes and spectrophotometric measurements. See, for example, Katerji et al. 2019 Oxid Med Cell Longev 1279250.

In some embodiments, contacting a cell with the bacterial strain composition or administering the bacterial strain composition results in an increase in production, amount, or activity of transcription factors (e.g., FOXO3a) that activate antioxidant enzymes, antioxidant enzymes (e.g., SOD1, CAT, GPx, GR, eNOS, nNOS), and reduced glutathione (GSH). An increase can be about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, at least a 5%, 10%, 20%, 30%, 40%, 50%, 60%, 70%, 80%, 90%, 100%, 150%, 200% or greater, at least about a 3%-15%, 10%-25%, 20% to 35%, 30% to 45%, 40%-55%, 50%-65%, 60%-75%, 70%-85%, 80%-95%, 90%-105%, 100%-115%, 105%-120%, 115%-130%, 125%-150%, 140%-160%, 155%-500% or greater, in the production, amount, or activity of transcription factors (e.g., FOXO3a), antioxidant enzymes (e.g., SOD1, CAT, GPx, GR, eNOS, nNOS), and reduced glutathione (GSH) when compared to an appropriate control. Methods to assay for the level of these transcription factors, and enzymes are known and include method known in the art to measure mRNA levels, including but not limited to, quantitative polymerase chain reaction (qPCR), reverse transcription following by qPCR (RT-qPCR), and Northern blot, and any method known in the art to measure protein levels, including but not limited to, Western blot and ELISA. Assays to measure the antioxidant status of the enzymes can also be used. See, for example, Katerji et al. 2019 Oxid Med Cell Longev 1279250. Methods to assay for GSH are known and include a recycling assay. See, for example, Forman et al. 2009 Mol Aspects Med 30(1-2):1-12.

In some embodiments, contacting a cell with the bacterial strain composition or administering the bacterial strain composition results in an increase in production, amount, or activity of vascular NO or constitutive NOS (i.e., eNOS and/or nNOS). As used herein, an “increase in” or “increasing” production, amount, or activity of vascular NO or constitutive NOS comprises any statistically significant increase, for example, an increase of about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, at least a 5%, 10%, 20%, 30%, 40%, 50%, 60%, 70%, 80%, 90%, 100%, 150%, 200% or greater, at least about a 3%-15%, 10%-25%, 20% to 35%, 30% to 45%, 40%-55%, 50%-65%, 60%-75%, 70%-85%, 80%-95%, 90%-105%, 100%-115%, 105%-120%, 115%-130%, 125%-150%, 140%-160%, 155%-500% or greater, in the production, amount, or activity of vascular NO or constitutive NOS when compared to an appropriate control. Methods to assay for the level of NO are known and include chemiluminescent detection and quantification of stable oxidation products using the Greiss reagent. See, for example, Weissman & Gross 2021 Curr Protoc Neurosci 7:7.13. Methods to assay for the level of constitutive NOS are known and include colorimetric ELISA assays. See, for example, Weissman & Gross 2021 Curr Protoc Neurosci 7:7.13.

In specific embodiments, administration of an effective amount of the bacterial strain composition comprising a bacterial strain of the invention can decrease the expression of a marker of oxidative stress compared to a proper control. Such decreases can include, for example, at least a 5%, 10%, 20%, 30%, 40%, 50%, 60%, 70%, 80%, 90%, or 100% decrease in the level of expression of a marker of oxidative stress, as measured by mRNA level, qPCR, protein level, ELISA, or any method known in the art, when compared to an appropriate control. The level of the oxidative stress marker can be measured as it relates to a proper control. As used herein a proper control includes but is not limited to, the expression level of a marker of oxidative stress in a corresponding sample from a subject that was not administered the bacterial strain composition comprising a bacterial strain of the invention, the expression level of a marker of oxidative stress in a sample from a subject prior to administration of the bacterial strain composition comprising a bacterial strain of the invention, or the expression level of a marker of oxidative stress in a standardized sample from a subject that was not administered the bacterial strain composition comprising a bacterial strain of the invention. One of skill in the art would be able to identify proper controls in order to measure an increase in the expression level of a marker of oxidative stress in a cell, a human subject, or in an agricultural animal.

### B. Methods of Reducing or Preventing Inflammation

Compositions and methods are provided herein for reducing or preventing inflammation in a cell or in a subject by contacting the cell with, or administering to the subject, a bacterial strain composition comprising a bacterial strain disclosed herein, and (in some embodiments) oleuropein or a plant, plant part, or extract thereof comprising oleuropein. Any cell may be contacted with a bacterial composition of the invention to reduce or prevent inflammation when compared to an appropriate control (e.g., a cell without contact with the bacterial strain composition). In some embodiments, the cell is a vascular cell (e.g., a vascular endothelial cell), a cardiac cell (e.g., a cardiomyocyte), a blood cell (e.g., a platelet, a leukocyte), an intestinal cell (e.g., an intestinal epithelial cell), a liver cell, a pancreatic cell, a kidney cell, a lung cell, an adipose cell, a neural cell, or a skin cell.

In some embodiments, administration of a bacterial strain composition comprising a bacterial strain of the invention can treat or prevent inflammation or an inflammatory disease. Administration of a bacterial strain composition provided herein can treat, ameliorate the symptoms, or prevent occurrence or progression of an inflammatory disease of the gastrointestinal tract, such as an inflammatory bowel disease (IBD), including, but not limited to, Crohn's disease and ulcerative colitis, in a subject. In some embodiments, administration of a bacterial strain composition comprising a bacterial strain of the invention can treat, ameliorate the symptoms, or prevent occurrence or progression of a systemic or focal inflammatory disorder outside the gastrointestinal tract, such as rheumatoid arthritis, systemic lupus erythematosus, multiple sclerosis, atopic dermatitis, and focal inflammatory disease in a subject. The inflammatory disorder can be any inflammatory disorder and need not be associated with gastrointestinal inflammation. The subject to be treated can be suffering from or at risk of developing any inflammatory disorder described herein, including, for example, be suffering from an IBD (e.g., Crohn's disease, ulcerative colitis), rheumatoid arthritis, systemic lupus erythematosus, multiple sclerosis, atopic dermatitis, and focal inflammatory disease.

In some embodiments, the reduction or decrease in inflammation may include stimulation of intestinal integrity; reduction of intestinal permeability; improvement of mucin synthesis, secretion, and/or quality; improvement of the maturation and differentiation of the intestinal epithelium; improvement of nutrient absorption; increase of the production of soluble factors that transfer antimicrobial activity; stimulation of, improvement of, or support of resistance to infection; support of cellular or humoral responses against viral or bacterial infection; increased cytotoxicity (both anti-viral and anti-tumor); support of systemic and/or mucosal vaccination responses; increase or support of cellular and/or humoral immunity; increase or support of natural immunity (including neutrophils, phagocytes, macrophages, and natural killer cell activity); increase or support of adaptive T and B cell immunity; stimulation of a helper T cell 1 (Th1) cytokine pattern (increased IL-1, IL-2, IFN-gamma, IL-12, TNF-alpha; human leukocyte antigen-Dr (HLA-Dr) expression); suppression of inflammation or production of systemic and mucosal inflammatory mediators (including cytokines and/or chemokines); reduction of sensitization by reducing total and/or allergen-specific IgE; reduction of the production of allergic cytokines; reduction of a Th2 supporting immunoglobulin profile; and combinations thereof when compared to an appropriate control (e.g., a sample from a subject prior to administration of a bacterial strain composition).

As used herein, the term “pro-inflammatory molecule” refers to a molecule that favors inflammation, such as a pro-inflammatory cytokine or enzyme that produces a pro-inflammatory cytokine (e.g., inducible nitric oxide synthase (iNOS), prostaglandin E2 synthase). As used herein, the term “pro-inflammatory cytokine” refers to an immunoregulatory cytokine that favors inflammation. Pro-inflammatory cytokines of the invention include nitric oxide produced by inducible nitric oxide synthase (iNOS), prostaglandin E2, IL1-alpha, IL1-beta, TNF-alpha, IL-2, IL-3, IL-6, IL-7, IL-9, IL-12, IL-17, IL-18, LT, LIF, CXCL10/IP-10, CCL2/MPC-1, MIP-1beta, matrix metaloproteinase-9, oncostatin, or IFN-alpha, IFN-beta, IFN-gamma.

As used herein, the term “anti-inflammatory molecule” refers to a molecule that exerts an anti-inflammatory effect, such as an anti-inflammatory cytokine or enzyme that produces an anti-inflammatory cytokine. As used herein, the term “anti-inflammatory cytokine” refers to a naturally occurring or recombinant protein, analog thereof or fragment thereof that elicits an anti-inflammatory response in a cell that has a receptor for that cytokine. Anti-inflammatory cytokines of the invention can be immunoregulatory molecules that control the pro-inflammatory cytokine response. Anti-inflammatory cytokines of the invention include interleukin (IL)-1 receptor antagonist, IL-4, IL-10, IL-11, and IL-13, IL-16, IFN-alpha, TGF-beta, G-CSF.

Methods and compositions also include those which decrease pro-inflammatory cytokine production or activity, which may decrease or prevent an inflammatory response, in a cell or a subject. As used herein, a decrease in the level of pro-inflammatory cytokine production or activity comprises any statistically significant decrease in the level of pro-inflammatory cytokine production or activity in a cell or a subject when compared to an appropriate control. Such decreases can include, for example, a decrease of about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or by at least 10%, 15%, 20%, 25%, 30%, 35%, 40%, 45%, 50%, 55%, 60%, 65%, 70%, 75%, 80%, 85%, 90%, 95%, or 100%, in the level of pro-inflammatory cytokine production or activity when compared to an appropriate control. Methods to assay for cytokine levels are known and include, for example, Leng S., et al. (2008) J Gerontol A Biol Sci Med Sci 63(8): 879-884. Methods to assay for the production of pro-inflammatory cytokines include multiplex bead assay, ELISPOT and flow cytometry. See, for example, Maecker et al. (2005) BMC Immunology 6:13.

In some embodiments, administration of the bacterial strain composition results in an increase in anti-inflammatory cytokine production or activity. As used herein, an “increase in” or “increasing” anti-inflammatory cytokine production comprises any statistically significant increase in the anti-inflammatory cytokine level when compared to an appropriate control. Such increases can include, for example, an increase of about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, at least a 5%, 10%, 20%, 30%, 40%, 50%, 60%, 70%, 80%, 90%, 100%, 150%, 200% or greater, at least about a 3%-15%, 10%-25%, 20% to 35%, 30% to 45%, 40%-55%, 50%-65%, 60%-75%, 70%-85%, 80%-95%, 90%-105%, 100%-115%, 105%-120%, 115%-130%, 125%-150%, 140%-160%, 155%-500% or greater, in the level of anti-inflammatory cytokine production or activity when compared to an appropriate control. Methods to assay for the level of anti-inflammatory cytokine level, are known. See, for example, Leng S., et al. (2008) J Gerontol A Biol Sci Med Sci 63(8): 879-884. Methods to assay for the production of anti-inflammatory cytokines include multiplex bead assay, ELISA, ELISPOT, qPCR, and flow cytometry. See, for example, Maecker et al. (2005) BMC Immunology 6:13.

Inflammatory cytokine production can also be measured by assaying the ratio of anti-inflammatory cytokine production to pro-inflammatory cytokine production. In specific aspects, the ratio of anti-inflammatory cytokine production to pro-inflammatory cytokine production is increased by about 1, 2, 3, 4, 5, 6, 7, 8, 9, 10, 20, 30, 40, 50, 60, 70, 80, 90, 100, 300, 600, 900, 1000 fold, or greater when compared to an appropriate control. In other aspects, the ratio of anti-inflammatory cytokine production to pro-inflammatory cytokine production is increased by about 1 to 5 fold, about 5 to 10 fold, about 10 to 20 fold, about 20 to 30 fold, about 30 to 40 fold, about 40 fold to 60 fold, about 60 fold to 80 fold, about 80 fold to about 100 fold, about 100 to 200 fold, about 200 fold to 300 fold, about 300 to 400 fold, about 400 to about 500 fold, about 500 to about 500 fold, about 500 fold to about 700 fold, about 700 fold to 800 fold, about 800 fold to about 1000 fold, or greater when compared to an appropriate control. Methods to determine the ratio of anti-inflammatory cytokine production to pro-inflammatory cytokine production can be found, for example, Leng S., et al. (2008) J Gerontol A Biol Sci Med Sci 63(8): 879-884. Methods to assay for the production of cytokines include multiplex bead assay, ELISA, ELISPOT, qPCR, and flow cytometry. See, for example, Maecker et al. (2005) BMC Immunology 6:13.

In specific embodiments, administration of an effective amount of the bacterial strain composition comprising a bacterial strain of the invention can decrease the expression of a marker of inflammation compared to a proper control. Such decreases can include, for example, at least a 5%, 10%, 20%, 30%, 40%, 50%, 60%, 70%, 80%, 90%, or 100% decrease in the level of expression of a marker of inflammation, as measured by mRNA level, qPCR, protein level, ELISA, LPS expression, or any method known in the art, when compared to an appropriate control. The level of the inflammation marker can be measured as it relates to a proper control. As used herein a proper control includes but is not limited to, the expression level of a marker of inflammation in a corresponding sample from a subject that was not administered the bacterial strain composition comprising a bacterial strain of the invention, the expression level of a marker of inflammation in a sample from a subject prior to administration of the bacterial strain composition comprising a bacterial strain of the invention, or the expression level of a marker of inflammation in a standardized sample from a subject that was not administered the bacterial strain composition comprising a bacterial strain of the invention. One of skill in the art would be able to identify proper controls in order to measure an increase in the expression level of a marker of inflammation in a cell, a human subject, or in an agricultural animal.

### C. Methods of Reducing or Inhibiting Platelet Aggregation

Compositions and methods are provided herein for reducing or inhibiting platelet aggregation in vitro, ex vivo, or in vivo by contacting a cell with, or administering to a subject, a bacterial strain composition comprising a bacterial strain disclosed herein, and (in some embodiments) oleuropein or a plant, plant part, or extract thereof comprising oleuropein. Any cell may be contacted with a bacterial composition of the invention to reduce or inhibit platelet aggregation when compared to an appropriate control (e.g., a cell without contact with the bacterial strain composition). In some embodiments, the cell is a vascular cell (e.g., a vascular endothelial cell), or a blood cell (e.g., a platelet).

In some embodiments, administration of a bacterial strain composition comprising a bacterial strain of the invention can reduce or inhibit platelet aggregation, or reduce or prevent thrombosis.

In some embodiments, the reduction or inhibition of platelet aggregation may include or involve a reduction in production or activity of thromboxane B2 (a chemically stable and inactive form of thromboxane A2), reduction in production, activity, or amount of thromboxane A2, a reduction in production or activity of cyclooxygenase-2 (COX-2), a reduction in production or activity of leukocyte inflammatory mediators (e.g., pro-inflammatory cytokines), an increase in production or activity of vascular nitric oxide (NO), an increase in activity of constitutive nitric oxide synthase [endothelial NOS (eNOS) and/or neuronal NOS (nNOS)], a decrease in activity of iNOS, and inhibition of cAMP-phosphodiesterases.

As used herein, a reduction or inhibition of platelet aggregation comprises any statistically significant decrease in the level of platelet aggregation in vitro, ex vivo, or in vivo when compared to an appropriate control. In some embodiments, contacting a cell with the bacterial strain composition or administering the bacterial strain composition results in a reduction or inhibition of about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or by at least 10%, 15%, 20%, 25%, 30%, 35%, 40%, 45%, 50%, 55%, 60%, 65%, 70%, 75%, 80%, 85%, 90%, 95%, or 100%, in the level of platelet aggregation when compared to an appropriate control. Methods to assay for platelet aggregation are known and include, for example, a platelet aggregation assay and a platelet aggregometry assay (Tsoupras et al. 2019 MethodsX 6:63-70).

In some embodiments, contacting a cell with the bacterial strain composition or administering the bacterial strain composition results in a reduction of about 10-100%, 20-100%, 30-100%, 40-100%, 50-100%, 60-100%, 70-100%, 80-100%, 20-90%, 30-90%, 40-90%, 50-90%, 60-90%, or 70-90% (e.g., by about 10-20%, 20-30%, 30-40%, 40-50%, 50-60%, 60-70%, 70-80%, 80-90%, or 90-100%), e.g., by about 10%, 15%, 20%, 25%, 30%, 35%, 40%, 45%, 50%, 55%, 60%, 65%, 70%, 75%, 80%, 85%, 90%, 95%, or 100%, or by at least 10%, 15%, 20%, 25%, 30%, 35%, 40%, 45%, 50%, 55%, 60%, 65%, 70%, 75%, 80%, 85%, 90%, 95%, or 100%, in production, amount, or activity of thromboxane B2, thromboxane A2, COX-2, leukocyte inflammatory mediators (e.g., pro-inflammatory cytokines), iNOS, or cAMP-phosphodiesterases. Methods to assay for these pro-platelet coagulation molecules are known and include, for example, measuring the levels of the gene product, such as measuring mRNA levels, e.g., quantitative polymerase chain reaction (qPCR), reverse transcription following by qPCR (RT-qPCR), and Northern blot, and measuring protein levels, including but not limited to, Western blot, ELISA, ELISPOT. See, for example, Leng S., et al. (2008) J Gerontol A Biol Sci Med Sci 63(8): 879-884; Maecker et al. (2005) BMC Immunology 6:13.

In some embodiments, administration of the bacterial strain composition results in an increase in production, amount, or activity of vascular NO or constitutive NOS (i.e., eNOS and/or nNOS). As used herein, an “increase in” or “increasing” production, amount, or activity of vascular NO or constitutive NOS comprises any statistically significant increase, for example, an increase of about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, at least a 5%, 10%, 20%, 30%, 40%, 50%, 60%, 70%, 80%, 90%, 100%, 150%, 200% or greater, at least about a 3%-15%, 10%-25%, 20% to 35%, 30% to 45%, 40%-55%, 50%-65%, 60%-75%, 70%-85%, 80%-95%, 90%-105%, 100%-115%, 105%-120%, 115%-130%, 125%-150%, 140%-160%, 155%-500% or greater, in the production, amount, or activity of vascular NO or constitutive NOS when compared to an appropriate control. Methods to assay for the level of NO are known and include chemiluminescent detection and quantification of stable oxidation products using the Greiss reagent. See, for example, Weissman & Gross 2021 Curr Protoc Neurosci 7:7.13. Methods to assay for the level of constitutive NOS are known and include colorimetric ELISA assays. See, for example, Weissman & Gross 2021 Curr Protoc Neurosci 7:7.13.

In specific embodiments, administration of an effective amount of the bacterial strain composition comprising a bacterial strain of the invention can decrease the expression of a marker of platelet aggregation compared to a proper control. Such decreases can include, for example, at least a 5%, 10%, 20%, 30%, 40%, 50%, 60%, 70%, 80%, 90%, or 100% decrease in the level of expression of a marker of platelet aggregation, as measured by mRNA level, qPCR, protein level, ELISA, LPS expression, or any method known in the art, when compared to an appropriate control. The level of the platelet aggregation marker can be measured as it relates to a proper control. As used herein a proper control includes but is not limited to, the expression level of a marker of platelet aggregation in a corresponding sample from a subject that was not administered the bacterial strain composition comprising a bacterial strain of the invention, the expression level of a marker of platelet aggregation in a sample from a subject prior to administration of the bacterial strain composition comprising a bacterial strain of the invention, or the expression level of a marker of platelet aggregation in a standardized sample from a subject that was not administered the bacterial strain composition comprising a bacterial strain of the invention. One of skill in the art would be able to identify proper controls in order to measure an increase in the expression level of a marker of platelet aggregation in a cell, a human subject, or in an agricultural animal.

### D. Methods for Treating or Preventing Disease or Condition in a Subject

Methods are provided herein for treating or preventing disease or condition associated with oxidative stress, inflammation, or platelet aggregation in a subject by administering a bacterial strain composition comprising a bacterial strain of the invention, and in some embodiments oleuropein or a plant, plant part, or extract thereof comprising a oleuropein. The disease or condition to be treated or prevented can include cardiovascular diseases (e.g., atherosclerosis, hypertension, coronary artery disease, myocardial infarction, angina, peripheral vascular disease, stroke), metabolic disorders (e.g., diabetes mellitus, glucose intolerance, dyslipidemia), neurodegenerative diseases (e.g., Alzheimer's disease), brain or spinal cord injury, inflammatory disorders (e.g., inflammatory bowel disease, Crohn's disease, ulcerative colitis, rheumatoid arthritis, systemic lupus erythematosus, multiple sclerosis, atopic dermatitis), and cancer (e.g., breast cancer, colon cancer, bladder cancer, brain cancer, leukemia, prostate cancer, renal cancer, thyroid cancer).

Methods of the present disclosure can treat or prevent cardiovascular disease. As used herein, “cardiovascular disease” refers to any disease that affects the heart or blood vessels, including atherosclerosis, hypertension, coronary artery disease, myocardial infarction, angina, peripheral vascular disease, and stroke. While not wishing to be bound to any theory or mechanism of action, it is believed that hydroxytyrosol provided by the methods disclosed herein can provide antioxidant effects, anti-inflammatory effects, anti-platelet aggregation effects, inhibition of endothelial activation including inhibition of expression or function of VCAM-1, inhibition of expression or function of extracellular regulated kinase-1/2, thereby inhibiting atherosclerosis and providing cardioprotective effect. See, for example, D'Angelo et al. 2020 Cells 9:1932.

The subject to be treated can be suffering from or at risk of developing a cardiovascular disease, including, for example, a subject with a genetic predisposition for the cardiovascular disease, or a subject having one or more risk factors, e.g., hypertension, dyslipidemia, diabetes, glucose intolerance, cigarette smoking, obesity, and sedentary lifestyle. Subjects can be treated that have a cardiovascular disease of any clinical or pathological stage.

A cardiovascular disease or condition can be considered treated if the subject undergoes a complete or partial response, which can be determined by objective criteria commonly used in clinical trials for the disease, e.g., as listed or accepted by the Food and Drug Administration or the American Heart Association. Non-limiting examples of such criteria include a reduction in the incidence and/or severity of symptoms and disease progression (e.g., atherosclerosis, calcification of arteries, blood pressure, blood cholesterol levels, blood triglyceride levels, blood glucose levels) as assessed by body fluid tests, functional tests, imaging tests, or pathology. Such decreases can include, for example, at least a 5%, 10%, 20%, 30%, 40%, 50%, 60%, 70%, 80%, 90%, or 100% decrease in the incidence and/or severity of symptoms and disease progression (e.g., atherosclerosis, calcification of arteries, blood pressure, blood cholesterol levels, blood triglyceride levels, blood glucose levels) as assessed by body fluid tests, functional tests, imaging tests, or pathology as compared to an appropriate control (e.g., a sample from a subject prior to administration of a bacterial strain composition).

As used herein a proper control includes but is not limited to, a corresponding sample from a subject that was not administered the bacterial strain composition comprising a bacterial strain of the invention, a subject prior to administration of the bacterial strain composition comprising a bacterial strain of the invention, or a standardized control.

The presently disclosed bacterial compositions can be administered along with (simultaneously or sequentially) any treatment known in the art, including but not limited to anticoagulation therapy, lipid-lowering medication, diabetic medication, antihypertensive medication, and diet and lifestyle modification.

Methods of the present disclosure can treat or prevent metabolic disease. As used herein, “metabolic disease” refers to any disease that affect metabolism of carbohydrate, glucose, or lipid, including diabetes mellitus, glucose intolerance, and dyslipidemia. Without wishing to be bound by theory, hydroxytyrosol can inhibit the SREBP-1c/FAS pathway in liver and skeletal muscle tissues, enhance antioxidant enzyme activities, normalize expression of mitochondrial complex subunits and mitochondrial fission marker Drp1, inhibit apoptosis activation, normalize serum glucose levels by enhancing glucose-induced insulin release and peripheral uptake of glucose, normalize serum triglyceride and cholesterol levels, and regulate metabolic pathway enzymes such as lipocalin 2, thereby preventing or treating metabolic disease. See, for example, Bulotta et al 2014 J. Translat. Med. 12:219.

The subject to be treated can be suffering from or at risk of developing a metabolic disease or condition, including, for example, a subject with a genetic predisposition for the disease or condition, or a subject having risk factors, e.g., dietary and lifestyle factors. Subjects can be treated that have a metabolic disease or condition of any clinical or pathological stage.

A metabolic disease or condition can be considered treated if the subject undergoes a complete or partial response, which can be determined by objective criteria commonly used in clinical trials for the disease, e.g., as listed or accepted by the Food and Drug Administration, the American Heart Association, or the American Diabetes Association. Non-limiting examples of such criteria include a reduction in the incidence and/or severity of symptoms and disease progression (e.g., blood cholesterol levels, blood triglyceride levels, blood glucose levels, presence or severity of complications e.g., diabetic retinopathy, diabetic nephropathy, diabetic vasculopathy, diabetic neuropathy, fatty liver) as assessed by biomarker levels, functional tests, imaging tests, or pathology. Such decreases can include, for example, at least a 5%, 10%, 20%, 30%, 40%, 50%, 60%, 70%, 80%, 90%, or 100% decrease in the incidence and/or severity of symptoms and disease progression (e.g., blood cholesterol levels, blood triglyceride levels, blood glucose levels, presence or severity of complications e.g., diabetic retinopathy, diabetic nephropathy, diabetic vasculopathy, diabetic neuropathy, fatty liver) as assessed by biomarker levels, functional tests, imaging tests, or pathology as compared to an appropriate control (e.g., a sample from a subject prior to administration of a bacterial strain composition).

As used herein a proper control includes but is not limited to, a corresponding sample from a subject that was not administered the bacterial strain composition comprising a bacterial strain of the invention, a subject prior to administration of the bacterial strain composition comprising a bacterial strain of the invention, or a standardized control.

The presently disclosed bacterial compositions can be administered along with (simultaneously or sequentially) any treatment known in the art, including but not limited to lipid-lowering medication, diabetic medication (oral medication, insulin injection), and diet and lifestyle modification.

Methods of the present disclosure can treat or prevent neurodegenerative diseases (e.g., Alzheimer's disease), and brain or spinal cord injury.

As used herein, the term “neurodegenerative disease” refers to a disease or condition resulting from progressive damage to cells in the central or peripheral nervous system, including Alzheimer's disease, Parkinson's disease, Huntington's disease, amyotrophic lateral sclerosis, and motor neuron disease. As used herein, the term “brain or spinal cord injury” refers to primary or secondary injury to the brain or the spinal cord, including traumatic injury, inflammatory injury, and degenerative injury.

The subject to be treated can be suffering from or at risk of developing a neurodegenerative disease or condition, including, for example, a subject with a genetic predisposition for the disease or condition, or a subject exposed to risk factors, e.g., substances that may increase risk of developing a neurodegenerative disease. Subjects can be treated that have a neurodegenerative disease or condition of any clinical or pathological stage.

A neurodegenerative disease or condition can be considered treated if the subject undergoes a complete or partial response, which can be determined by objective criteria commonly used in clinical trials for the disease, e.g., as listed or accepted by the Food and Drug Administration, the Alzheimer's Association, or the National Institute of Health. Non-limiting examples of such criteria include a reduction in the incidence and/or severity of symptoms and disease progression as assessed by biomarker levels, functional tests, imaging tests, or pathology. Such decreases can include, for example, at least a 5%, 10%, 20%, 30%, 40%, 50%, 60%, 70%, 80%, 90%, or 100% decrease in the incidence and/or severity of symptoms, disease progression as assessed by biomarker levels, functional tests, imaging tests, or pathology as compared to an appropriate control (e.g., a sample from a subject prior to administration of a bacterial strain composition).

As used herein a proper control includes but is not limited to, a corresponding sample from a subject that was not administered the bacterial strain composition comprising a bacterial strain of the invention, a subject prior to administration of the bacterial strain composition comprising a bacterial strain of the invention, or a standardized control.

The presently disclosed bacterial compositions can be administered along with (simultaneously or sequentially) any treatment known in the art, including but not limited to medication for neurodegenerative disease, physical therapy, and cognitive therapy.

Methods of the present disclosure can treat or prevent cancer. As used herein, the term “cancer” should be understood to encompass any neoplastic disease (whether invasive or metastatic) which is characterized by abnormal and uncontrolled cell division causing malignant growth or tumor. Cancers that may be treated with the presently disclosed compositions and methods include tumors that are not vascularized, or not yet substantially vascularized, as well as vascularized tumors. The cancers may include non-solid tumors (such as hematological tumors, for example, leukemias and lymphomas) or may include solid tumors. Non-limiting examples of types of cancers to be treated with the presently disclosed compositions and methods include, but are not limited to, carcinoma, blastoma, and sarcoma, and certain leukemia or lymphoid malignancies, benign and malignant tumors, and malignancies such as sarcomas, carcinomas, and melanomas. Other non-limiting examples include breast cancer, prostate cancer, ovarian cancer, cervical cancer, skin cancer, pancreatic cancer, colorectal cancer, renal cancer, bladder cancer, liver cancer, brain cancer, lymphoma, leukemia, thyroid cancer, and lung cancer.

Without wishing to be bound by theory, hydroxytyrosol provided by the methods can provide tissue protective effects and anti-tumor effects by exerting antioxidant and anti-inflammatory functions, thereby treating or preventing cancer. See, for example, Bulotta et al 2014 J. Translat. Med. 12:219.

The subject to be treated can be suffering from or at risk of developing cancer, including, for example, a subject with a genetic predisposition for cancer, or a subject exposed to risk factors, e.g., carcinogens. Subjects can be treated that have cancer of any clinical or pathological stage.

A disease or condition can be considered treated if the subject undergoes a complete or partial response, which can be determined by objective criteria commonly used in clinical trials for the disease, e.g., as listed or accepted by the Food and Drug Administration and the National Cancer Institute. Non-limiting examples of such criteria include a change (e.g., reduction) in the number of tumors, the tumor size, or the tumor cell numbers, metastasis of cancer, or cancer biomarker levels, as assessed by pathology, imaging tests, or marker level quantification. Such decreases can include, for example, at least a 5%, 10%, 20%, 30%, 40%, 50%, 60%, 70%, 80%, 90%, or 100% decrease in the incidence and/or severity of symptoms, disease progression (e.g., number of tumors, the tumor size, or the tumor cell numbers, metastasis of cancer, or cancer biomarker levels, as assessed by pathology, imaging tests, or marker level quantification as compared to an appropriate control (e.g., a sample from a subject prior to administration of a bacterial strain composition).

As used herein a proper control includes but is not limited to, a corresponding sample from a subject that was not administered the bacterial strain composition comprising a bacterial strain of the invention, a subject prior to administration of the bacterial strain composition comprising a bacterial strain of the invention, or a standardized control.

The presently disclosed bacterial compositions can be administered along with (simultaneously or sequentially) any treatment known in the art, including but not limited to lifestyle modification to avoid carcinogen exposure, surgery, radiation therapy, and chemotherapeutic drugs.

The following examples are offered by way of illustration and not by way of limitation.

### EXPERIMENTAL

### Example 1: Isolation and Genomic Characterization of Lactobacillus pentoses Strain OL79

### Materials and Methods

Lactobacillus pentoses strain OL79 was identified from isolates recovered from naturally fermented olives for its ability to convert oleuropein to hydroxytyrosol. High molecular weight genomic DNA was extracted for genome sequencing from 1 ml of overnight culture using phenol:chloroform:isoamyl alcohol with an ethanol precipitation. A short-read shotgun genomic library was prepared with Nextera DNA Flex (Illumina, San Diego, Calif.). The library was sequenced for 151 cycles from each end on a iSeq 100 (Illumina). A long-read genomic library was prepared with Ligation Sequencing Kit SQK-LSK109 (Oxford Nanopore Technologies, Oxford, UK). The library was sequenced for 24 hours on a flongle flow cell within a MinION Mk1B sequencer (Oxford Nanopore Technologies). Fastq files were analyzed with FastQC and USEARCH (Babraham Bioinformatics—FastQC; Edgar R C. 2010 Bioinformatics 26:2460-2461). The genome was assembled using Unicycler with bold settings (Wick et al. 2017 PLOS Computational Biology 13:e1005595). Genome statistics were calculated using quast (Gurevich et al. 2013 Bioinformatics 29:1072-1075). The genome was annotated in PATRIC using RASTtk (Brettin et al. 2015 Scientific Reports 5:8365).

To taxonomically identify OL79, the completed genome assembly was submitted to the Type Strain Genome Server (Meier-Kolthoff & Goker 2019 Nat Commun 10:2182). Genome completeness was analyzed using checkM (Parks et al. 2015 Genome Res 25:1043-1055).

Screening for acquired antibiotic resistance genes in the genome was done using ResFinder 3.2 (minimum 90% identity, 60% length) and CARD 3.0.9 (Znkari et al. 2012 Nucleic Acids Res 38:W647-W651; J Antimicrob Chemother 67:2640-2644; Alcock et al. 2020 Nucleic Acids Res 48:D517-D525). Secondary metabolites were analyzed using BAGEL4 and antiSMASH (de Jong et al. 2010 Nucleic Acids Res 38:W647-W651; Blin et al. 2019 Nucleic Acids Res 47:W81-W87). Metabolic mapping was done using GhostKoala (Kanehisa et al. J Mol Biol 428:726-731).

### Results and Discussion

There were 823,401 paired Illumina reads with an average length of 147 bp (64-fold coverage) with an average phred score of 34.9 (99.97% accurate). There were 47,060 long reads with an average length of 4,910 bp (61-fold coverage). The final genome assembly consists of 1 circular chromosomal contig (3,538,645 bp) and 10 circular plasmids covering a total length of 3,807,559 bp. The genome was predicted to be 99.38% complete. Genome assembly and annotation characteristics are summarized in Table 1 below. Plasmid characteristics are summarized in Table 2 below.

TABLE 1
Genome assembly and annotation characteristics

| Characteristic | OL79 |
|---|---|
| Size (bp) | 3,807,559 |
| Contigs | 11 |
| Circular plasmids | 10 |
| % GC | 46.07% |
| CDS | 3,591 |
| Completeness (%) | 99.38% |

TABLE 2
Plasmid characteristics

| | Length | | Normalized |
|---|---|---|---|
| Contig name | (bp) | CDS | coverage |
| 2 | 57,566 | 57 | 1.6x |
| 3 | 53,103 | 67 | 1.7x |
| 4 | 53,004 | 56 | 2.5x |
| 5 | 46,044 | 45 | 4.9x |
| 6 | 21,227 | 26 | 4.6x |
| 7 | 12,726 | 13 | 4.8x |
| 8 | 11,430 | 10 | 6.3x |
| 9 | 9,588 | 12 | 4.2x |
| 10 | 2,411 | 2 | 15.7x |
| 11 | 1,815 | 2 | 16.1x |

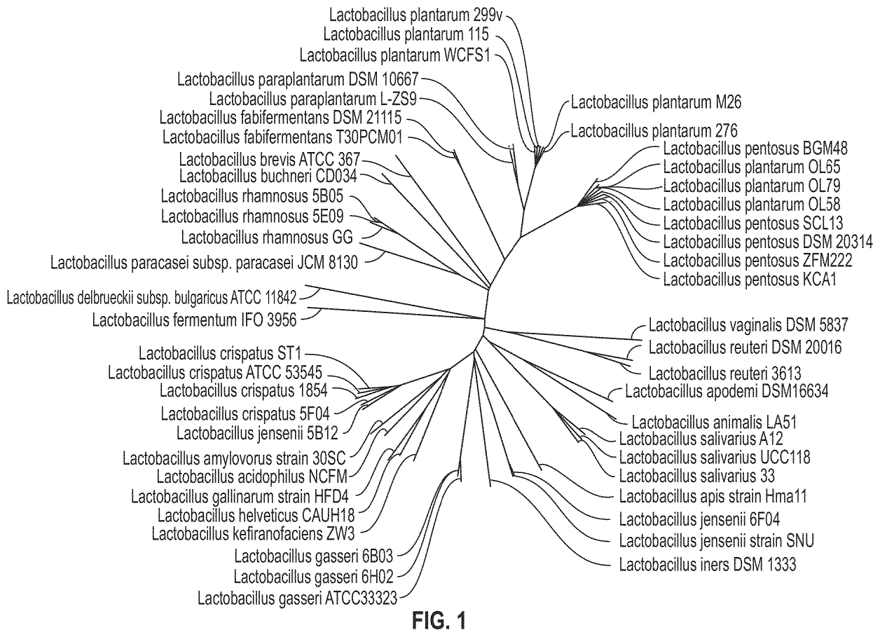

The Type Strain Genome Server taxonomically identified OL79 as Lactobacillus pentosus. The phylogenetic relationship with other strains is shown in FIG. 1. The recently updated taxonomic species for OL79 is Lactiplantibacillus pentosus, although most databases have not been updated and there are no regulatory changes (Zheng et al. 2020 J System Evol Microbiol).

As shown in Tables 3 and 4, no antibiotic resistance genes were detected in the genome using ResFinder or CARD. As shown in Table 5, Minimum Inhibitory Concentrations (MICs) for relevant antibiotics demonstrated susceptibility consistent with EFSA cutoff values. As shown in Table 6, one secondary metabolite, a Type III polyketide synthase, was identified using antiSMASH. BAGEL identified a pediocin on the chromosomal contig and a bovicin-like peptide on the circular plasmid contig 3 as secondary metabolites.

TABLE 3
ResFinder results of antibiotic resistance
for Lactobacillus pentosus strain OL79

| Microbial agent | Resistance genes | E-value |
|---|---|---|
| Aminoglycoside | None found | N/A |
| Beta-lactam | None found | N/A |
| Colistin | None found | N/A |
| Fluoroquinolone | None found | N/A |
| Fosfomycin | None found | N/A |
| Fusidic acid | None found | N/A |
| Glycopeptide | None found | N/A |
| Macrolide, lincosamide, | None found | N/A |
| streptogramin B | | |
| Nitroimidazole | None found | N/A |
| Oxazolidinone | None found | N/A |
| Phenicol | None found | N/A |
| Rifampicin | None found | N/A |
| Sulphonamide | None found | N/A |
| Tetracycline | None found | N/A |
| Trimethoprim | None found | N/A |

TABLE 4
CARD results of antibiotic resistance for
Lactobacillus pentosus strain OL79

| Strictness | Number of hits |
|---|---|
| Perfect | 0 |
| Strict | 0 |

TABLE 5
Bacterial minimum inhibitory concentration (MIC) cutoff
values for Lactobacillus pentosus strain OL79

| | EFSA Break | |
|---|---|---|
| | Point (mg/L) | Susceptibility |
| Gentamycin | 16 | sensitive |
| Ampicillin | 2 | sensitive |
| Erythromycin | 1 | sensitive |
| Chloramphenicol | 8 | sensitive |
| Clindamycin | 4 | sensitive |
| Tetracycline | 32 | sensitive |
| Kanamycin | 64 | sensitive |

TABLE 6
Secondary metabolite results for
Lactobacillus pentosus strain OL79

| Hit | Location (contig: location) |
|---|---|
| Type III polyketide | Contig 1: 1,550,027-1,591,196 |
| synthase (T3PKS) | |
| Pediocin | Contig 1: 163,850-184,150 |
| Bovicin-like | Contig 3: 27,725-47,854 |

Lactobacillus plantarum group strain OL79 was found to be free of antibiotic resistance genes. Originally isolated based on its ability to transform oleuropein into hydroxytyrosol, Lactobacillus pentosus OL79 also encodes secondary metabolite biosynthetic genes, including a type III polyketide synthase, a pediocin, and bovicin-like peptide (Table 6).

Unlike other Lactobacillus plantarum group strains, strain OL79 does not encode any glutamate decarboxylases, i.e., enzymes that consume L-glutamate to produce 4-aminobutanoate (GABA), an inhibitory neurotransmitter in the human cortex.

Remarkably, OL79 encodes ten circular plasmids which range in size from 1,815 bp to 57,566 bp (Table 2). The 53,103 bp plasmid encodes the bovicin-like peptide. There are genes for carbohydrate metabolism and arsenate resistance. GhostKoala did not reveal any metabolic pathways on the plasmids.

### Example 2: Hemolytic Activity and Virulence Genes in Lactobacillus Pentoses Strain OL79

### Hemolytic Activity

The genome of Lactobacillus pentosus strain OL79 was analyzed using Geneious 10.2.6. No hemolysin complete genes were detected. Moreover, plating on blood agar did not demonstrate the presence of any hemolytic activity.

### Virulence Genes

A search for homologs of known virulence factors, which enhance a bacteria's potential to cause disease, was performed in PATRIC (Davis et al. 2016 Front Microbiol 7). The virulence factors from three databases were analyzed: Victors with 5304 virulence factors (Sayers et al. 2019 Nucleic Acids Res 47:D693-D700), Virulence Factor DataBase (VFDB) with 3694 curated virulence genes (Liu et al. 2019 Nucleic Acids Res 47:D687-D692) and PATRIC_VF with 1293 genes. No homologs of known virulence genes were detected in Lactobacillus pentosus strain OL79.

### Example 3: Gastrointestinal Performance of Lactobacillus pentosus Strain OL79

Lactobacillus pentosus strain OL79 was identified from the studies described in Example 1 as a unique strain that can convert oleuropein to hydroxytyrosol.

Lactobacillus are naturally found in the gastrointestinal tract of humans and animals and many species have a long safe history of use in food preservation and production with multiple defined health benefits. Survival in the gastrointestinal tract requires a variety of attributes, including acid tolerance and the ability to withstand bile salts. In vitro assays have demonstrated that Lactobacillus pentosus strain OL79 is largely resistant to low pH conditions (pH 3.0) and survives in the presence of physiological concentrations of bile salts for extended periods of time. Table 7 below summarizes the findings.

TABLE 7
Acid and Bile Tolerance of Lactobacillus pentosus strain OL79

| | |
|---|---|
| Acid | No loss of survival in medium containing hydrochloric |
| Tolerance | acid at pH 3.0 for 3 hr at 37° C. |
| Bile | Greater than 97% survival in 0.3% bile salt containing |
| Tolerance | medium for 3 hr at 37° C. |

### Example 4: Conversion of Oleuropein to Hydroxytyrosol by Lactobacillus Pentoses Strain OL79

Overnight culture of Lactobacillus pentosus strain OL79 was pelleted at 8000×g for 10 min. Cells were washed 1× with sterile peptone and spun at 8000×g for 10 min. The pellet was resuspended in 10 ml minimal media containing 20 mM oleuropein. Culture was incubated for 18 hr at 37° C. in the anaerobic chamber. At 18 hr, the culture supernatant was filter sterilized and assessed by Ultra High Performance Liquid Chromatography (HPLC) for detection of hydroxytyrosol as well as reduction in oleuropein

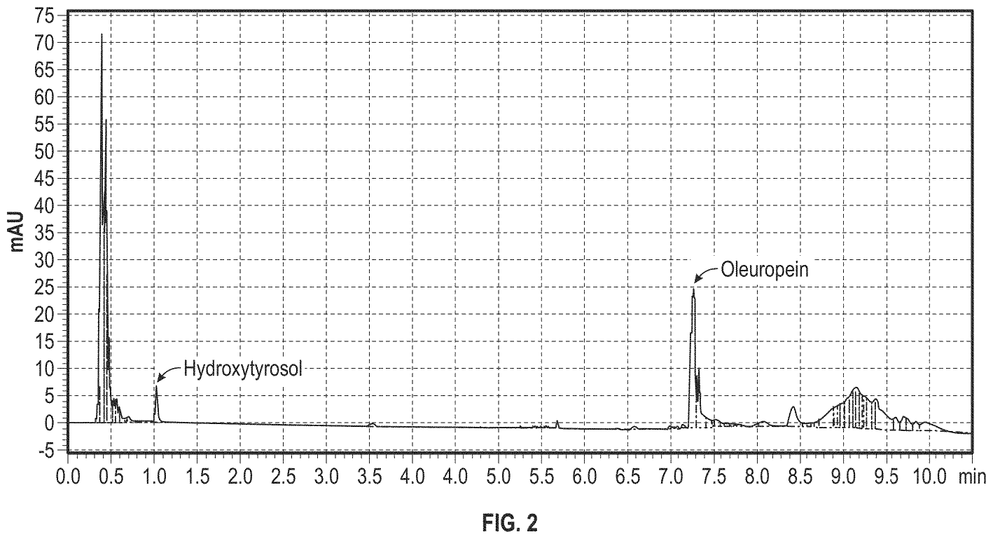

As shown in FIG. 2, the HPLC chromatograph showed detection of hydroxytyrosol (retention time at about 1.0 minute) and reduction in oleuropein (retention time at about 7.3 minutes). The findings support that Lactobacillus pentosus strain OL79 is active and converts oleuropein to hydroxytyrosol.

All citations to references, including, for example, citations to patents, published patent applications, and articles, are herein incorporated by reference in their entirety.

The section headings used herein are for organizational purposes only and are not to be construed as limiting the subject matter described in any way.

1. A bacterial strain composition comprising a bacterial cell of Lactobacillus pentosus strain OL79 or an active variant thereof,
wherein said Lactobacillus pentosus strain OL79 cell is present at about 10⁶ CFU/gram to about 10¹⁰ CFU/gram or at about 10⁶ CFU/ml to about 10¹⁰ CFU/ml,
wherein said bacterial strain composition further comprises oleuropein, and/or
wherein said bacterial strain composition is a pharmaceutical composition and further comprises a pharmaceutically acceptable carrier.

2. (canceled)

3. The bacterial strain composition of claim 1, wherein said composition comprises at least one plant, plant part, or extract thereof comprising said oleuropein.

4. The bacterial strain composition of claim 3, wherein said at least one plant, plant part, or extract thereof comprises an olive plant, olive plant part, or extract thereof.

5. The bacterial strain composition of claim 1, wherein an effective amount of said bacterial strain composition increases conversion of oleuropein to hydroxytyrosol, reduces oxidative stress, inflammation, and/or platelet aggregation, and/or treats in a subject cardiovascular disease, a metabolic disease, an inflammatory disease, a neurodegenerative disease, or a cancer, relative to the absence of said effective amount of said bacterial strain composition.

6.-8. (canceled)

9. The bacterial strain composition of claim 5, wherein an effective amount of said bacterial strain comprises about 10⁶ CFU/gram to about 10¹⁰ CFU/gram or about 10⁶ CFU/ml to about 10¹⁰ CFU/ml of said Lactobacillus pentosus strain OL79 or the active variant thereof.

10. The bacterial strain composition of claim 1, wherein said bacterial strain composition comprises a capsule, gel, paste, cream, ointment, tablet, powder, or liquid.

11.-15. (canceled)

16. A method of increasing hydroxytyrosol production, said method comprising contacting oleuropein with an effective amount of a bacterial strain composition comprising a bacterial cell of Lactobacillus pentosus strain OL79 or an active variant thereof.

17. The method of claim 16, comprising contacting at least one plant, plant part, or extract thereof comprising said oleuropein with said effective amount of said bacterial strain.

18. The method of claim 17, wherein said at least one plant, plant part, or extract thereof comprises olive plant, olive plant part, or extract thereof.

19. A method of increasing an amount of hydroxytyrosol in a subject, said method comprising administering to a subject an effective amount of a bacterial strain composition comprising a bacterial cell of Lactobacillus pentosus strain OL79 or an active variant thereof.

20. The method of claim 19, wherein said subject is administered oleuropein.

21. (canceled)

22. The method of claim 20, wherein said subject is administered at least one plant, plant part, or extract thereof comprising said oleuropein.

23. The method of claim 22, wherein said at least one plant, plant part, or extract thereof comprises olive plant, olive plant part, or extract thereof.

24. The method of claim 19, wherein said bacterial composition is administered orally or dermally.

25. The method of claim 19, wherein said effective amount of said bacterial strain composition comprises about 10⁶ CFU/gram to about 10¹⁰ CFU/gram or about 10⁶ CFU/ml to about 10¹⁰ CFU/ml of said Lactobacillus pentosus strain OL79 or the active variant thereof.

26. (canceled)

27. A method for reducing or preventing oxidative stress, inflammation, and/or platelet aggregation in a subject, said method comprising administering to said subject an effective amount of a bacterial strain composition of claim 1.

28. (canceled)

29. The method of claim 27, comprising treating a disease or a condition associated with oxidative stress, inflammation, or platelet aggregation in a subject.

30. The method of claim 29, wherein the disease or the condition is a cardiovascular disease, a metabolic disease, an inflammatory disease, a neurodegenerative disease, or a cancer.

31.-34. (canceled)

35. The method of claim 27, wherein an amount of hydroxytyrosol is increased in the subject relative to the absence of said effective amount of said pharmaceutical composition.

36.-38. (canceled)

39. A method of reducing oxidative stress, reducing inflammation, and/or reducing platelet aggregation, said method comprising contacting a cell with an effective amount of a bacterial strain composition of claim 1.

40.-41. (canceled)
